(19) United States
(12) Patent Application Publication
(10) Pub. No.: US 2007/0117836 A1
(43) Pub. Date: May 24, 2007

(54) NAPTHALIMIDE DERIVATIVES, METHODS FOR THEIR PRODUCTION AND PHARMACEUTICAL COMPOSITIONS THEREFROM

(76) Inventors: Eric VAN QUAQUEBEKE, WOLUWE ST. LAMBERT (BE); Gentiane SIMON, Bruxelles (BE); Laurent VAN DEN HOVE, Bruxelles (BE); Robert KISS, St-Pieters Leeuw (BE); Francis DARRO, Uccle (BE)

Correspondence Address: CLARK & ELBING LLP, 101 FEDERAL STREET, BOSTON, MA, 02110, US

(21) Appl. No.: 11/556,571

(22) Filed: Nov. 3, 2006

Related U.S. Application Data
(63) Continuation-in-part of application No. PCT/BE05/00069, filed on May 4, 2005.
(60) Provisional application No. 60/568,469, filed on May 5, 2004.

(30) Foreign Application Priority Data
May 5, 2004 (EP) 04447114.2

(51) Int. Cl. A61K 31/473; C07D 221/04

(52) U.S. Cl. 514/296; 546/98

(57) ABSTRACT

Novel substituted naphthalimide derivatives, pharmaceutically acceptable salts thereof and solvates thereof, are useful for making pharmaceutical compositions for the treatment of cell proliferative diseases such as cancer. The invention also provides methods for making such derivatives.

### CROSS REFERENCE TO RELATED APPLICATIONS

This application is a continuation-in-part of International Application No. PCT/BE2005/000069, filed on May 4, 2005, which was published in English under PCT Article 21(2) as WO 05/105,753, and which claims the benefit of European patent application No. 04447114.2 filed on May 5, 2004 and of U.S. provisional patent application No. 60/568,469 filed on May 5, 2004, the disclosures of which are incorporated by reference in their entirety.

### FIELD OF THE INVENTION

The present invention relates to novel substituted naphthalimides and 1,2-dihydro-3H-dibenzisoquinoline-1,3-dione derivatives, methods for their production and their pharmaceutical uses as anti-tumor agents, in particular in the form of pharmaceutical compositions including them as active principles in the prevention and/or treatment of various forms of cancer.

### BACKGROUND OF THE INVENTION

Various kinds of substituted naphthalimides are known in the art as having anti-tumour effect or other useful biological activity.

For instance, U.S. Pat. Nos. 3,935,227 and 3,940,398 disclose compounds having the following formula: 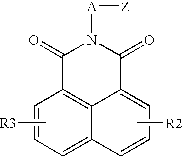
wherein R₂ and R₃ are each independently selected from the group consisting of hydrogen, halogen, lower alkyl, lower alkoxy, lower alkylthio, nitro, cyano, amino, and trifluoromethyl; A is a straight or branched chain alkylene of 1 to 8 carbons; and Z is an optionally substituted piperidinyl or piperazinyl group; or the pharmaceutically acceptable acid addition salts thereof, as exhibiting antidepressant activity and being also useful as anti-inflammatory agents.

U.S. Pat. No. 4,146,720 discloses compounds having the general formula:
wherein R₂ is nitro and R₁ is 2-diethylaminoethyl, 2-dimethylaminoethyl, 2-(N-pyrrolidino)ethyl 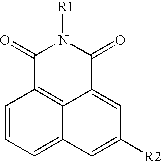
or 2-(N-piperidino)ethyl, as compounds being very active cytostatic agents as well as raticide and muricide agents. U.S. Pat. No. 4,204,063 discloses a family of compounds having the above general formula, wherein R₂ is alkyl, hydroxyl, alkoxy, halogen, amino, sulfonic acid, nitro, NHCOOC₂H₅, acetylamino or acetoxy, and wherein R₁ is an alkylene having one to three carbons and bonded to a nitrogen-containing group, such as dimethylamino, diethylamino, pyrrolidino, piperidino, N-methylpiperazino, morpholino or ureyl, and derivatives thereof, such as the salts thereof with pharmacologically acceptable acids, N-oxides and quaternary ammonium salts, as having great biological interest as anti-tumor agents.

U.S. Pat. No. 4,499,266, U.S. Pat. No. 4,594,346, U.S. Pat. No. 4,614,820 and U.S. Pat. No. 4,665,071 all disclose 3,6-dinitro-1,8-naphthalimide compounds which are useful antimicrobial agents and antitumor agents.

U.S. Pat. No. 5,183,821 discloses a method of treating a patient with leukemia or solid tumors, comprising administering to said patient 1-64 mg/kg of body weight of N-(2-dimethylaminoethyl)-3-amino-1,8-naphthalimide (amonafide), i.e. a compound having the above general formula wherein R₂ is amino.

U.S. Pat. No. 5,420,137 discloses specific salts of amonafide, especially the monohydrochloride and the monomethanesulfonate. WO 04/004716 discloses comprising a naphthalimide as a diammonium salt such as amonafide dimesylate or dihydrochloride.

Amonafide is an isoquinolinedione derivative which has undergone extensive tests for its anti-tumour activity. Among its biological activities, amonafide was reportedly shown to be a DNA intercalating agent and to inhibit topoisomerase II (e.g. etoposide) and to result in intercalator-stabilised-topoisomerase II-DNA clearable complex formation. In view of its DNA topoisomerase inhibiting effect, amonafide was taught in U.S. Pat. No. 6,037,326 to be also useful for reducing hair growth. Although the level of activity found for amonafide was and continues to be of high interest, this material does have significant deficiencies which indicate the continuing need for agents with improved properties. In the first place, amonafide was found to be too toxic for some patients: in particular it has produced substantial myelotoxicity leading to some deaths in patients receiving five daily doses of the drug. In addition, it was shown that amonafide had only moderate activity in leukemia models in mice. Also, it was shown that amonafide has no activity in human tumour xenografts in mice with colon, lung and mammary cancers. Thus, while amonafide shows significant biological activity, it does not have a substantially broad spectrum of activity in murine tumour models. Ajani et al. in Invest New Drugs (1988) 6:79-83 has shown that amonafide has poor activity when tested in primary human solid tumours in vitro.

Combinations of substituted naphthalimides with other therapeutic agents are also known in the art. For instance, U.S. Pat. No. 5,057,304 discloses an antitumor composition consisting essentially of an effective amount of a cancerostatic agent such as amonafide or mitonafide and an effective amount of a compound which reinforces the antitumor action of the cancerostatic agent.

U.S. Pat. No. 6,630,173 discloses treating a host with a cellular proliferative disease, comprising said host with a naphthalimide comprising an amonafide in conjunction with an antiproliferative agent comprising cisplatin. U.S. Pat. No. 6,423,696 discloses a composition comprising amonafide and an inhibitor interacting with N-acetyl transferase (NAT) to inhibit NAT from acetylating the arylamine group present in amonafide.

U.S. Pat. No. 5,554,622 discloses certain asymmetrically substituted bisnaphthalimides which activate non-specific immune cells which kill tumor cells and therefore may be used in pharmaceutical compositions for treating cancer.

Although the clinical activity of antiproliferative agents such as amonafide against certain forms of cancers can be shown,. improvement in tumor response rates, duration of response and ultimately patient survival are still sought. There is also a need in the art for improving the efficacy of antiproliferative treatments in humans by providing suitable combinations of new drugs with conventional antineoplastic agents.

In view of the above-mentioned shortcomings of amonafide and similar drugs available heretofore, the present inventors searched for amonafide derivatives which could demonstrate to be more effective anti-cancer agents. Specifically, they searched for compounds having the following characteristics:
1) increased tumor cell cytotoxic potency;
2) minimal, if any, cross resistance with multidrug resistant tumor cells;
3) relativity low cytotoxic potency in normal heart cells;
4) activity in a wide range of malignant tumors, especially solid tumors, hematological tumors, and leukemia; and
5) reduced myelotoxicity in humans at the tumor cell cytotoxic dosage.

As a result of their research, the present inventors have developed the following compounds, methods and compositions meeting these objectives.

### SUMMARY OF THE INVENTION

In a first embodiment, the invention provides a family of substituted naphthalimide (isoquinolinedione) derivatives represented by the general formula (I) 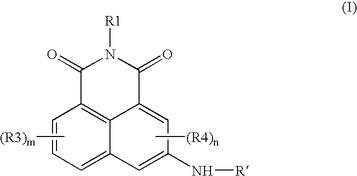
herein:
R₁ is a radical selected from the group consisting of monoalkylaminoalkyl, dialkylaminoalkyl, alkenylaminoalkyl, alkynylaminoalkyl, arylaminoalkyl, Het¹aminoalkyl, Het¹alkylaminoalkyl, Het¹ arylaminoalkyl, Het¹carbonylaminoalkyl, Het¹thiocarbonylaminoalkyl, alkylcarbonylaminoalkyl, alkenylcarbonyl-aminoalkyl, alkynylcarbonylaminoalkyl, arylcarbonylaminoalkyl, alkylthio-alkyl, arylthioalkyl, alkyloxyalkyl, aryloxyalkyl, alkylureylalkyl, alkenylureyl-alkyl, alkynylureylalkyl, arylureylalkyl, Het¹ureylalkyl, alkylcarbonylureyl-alkyl, alkenylcarbonylureylalkyl, alkynylcarbonylureylalkyl and arylureyl-alkyl, wherein one or more carbon atoms of said radical are optionally substituted by one or more substituents independently selected from the group consisting of oxo, alkyl, arylalkyl, aryl, Het¹, Het², cycloalkyl, alkyloxycarbonyl, carboxyl, aminocarbonyl, mono- or di(alkyl)aminocarbonyl, aminosulfonyl, alkyl-S(═O)ₜ, hydroxy, cyano, halogen, amino, nitro, mono- and disubstituted amino wherein the substituent(s) of the amino group is (are) independently selected from the group consisting of alkyl, aryl, arylalkyl, aryloxy, arylamino, arylthio, aryloxyalkyl, arylaminoalkyl, arylalkoxy, alkylthio, alkoxy, aryloxyalkoxy, arylaminoalkoxy, arylalkylamino, aryloxyalkylamino, arylaminoalkylamino, arylthioalkoxy, arylthioalkylamino, aralkylthio, aryloxyalkylthio, arylaminoalkylthio, arylthioalkylthio, alkylamino, cycloalkyl, cycloalkylalkyl, Het¹, Het², Het¹alkyl, Het²alkyl, Het¹amino, Het²amino, Het¹alkylamino, Het²alkylamino, Het¹thio, Het²thio, Het¹alkylthio, Het²alkylthio, Het¹oxy, Het²oxy, OR¹¹, SR¹¹, SO₂NR¹¹R¹², SO₂N(OH)R¹¹, CN, CR¹¹═NR¹², S(O)R¹¹, SO₂R¹¹, CR¹¹═N(OR¹²), N₃, NO₂, NR¹¹R¹², N(OH)R¹¹, C(O)R¹¹, C(S)R¹¹, CO₂R¹¹, C(O)SR¹¹, C(O)NR¹¹R¹², C(S)NR¹¹R¹², C(O)N(OH)R¹², C(S)N(OH)R¹¹, NR¹¹C(O)R¹², NR¹¹C(S)R¹², N(OH)C(O)R¹², N(OH)C(S)R¹¹, NR¹¹CO₂R¹², NR¹¹c(o)NR¹²R¹³, and NR¹¹C(S)NR¹²R¹³, N(OH)CO₂R¹¹, NR¹¹C(O)SR¹², N(OH)C(O)NR¹¹R¹², N(OH)C(S)NR¹¹R¹², NR¹¹C(O)N(OH)R¹², NR¹¹C(S)N-(OH)R¹², NR¹¹SO₂R¹², NHSO₂NR¹¹R¹², NR¹¹SO₂NHR¹² and P(O)(OR¹¹)(OR¹²), wherein t is 1 or 2, and wherein R¹¹, R¹² and R¹³ are each independently selected from the group consisting of hydrogen, alkyl, alkenyl, and alkynyl;
each of the substituents R₃ and R₄ is independently selected from the group consisting of hydrogen, halogen, C₁₋₇ alkyl, C₁₋₇ alkoxy, C₁₋₇ alkylthio, nitro, cyano, amino, protected amino and halo C₁₋₇ alkyl;
m is the number of substituents R₃ and is an integer ranging from 0 to 3;
n is the number of substituents R₄ and is an integer ranging from 0 to 2; and
R′ is a radical selected from the group consisting of C₂₋₇ alkylcarbonyl, alkenylcarbonyl, alkynylcarbonyl, arylcarbonyl, alkyloxycarbonyl, alkenyloxycarbonyl, aryloxycarbonyl, aryloxyalkylcarbonyl, cycloalkylcarbonyl, arylalkylcarbonyl, Het¹carbonyl, Het¹alkylcarbonyl, Het¹oxycarbonyl, Het¹alkyloxycarbonyl, alkylthiocarbonyl, alkenylthiocarbonyl, alkynylthiocarbonyl, arylthiocarbonyl, arylalkylthiocarbonyl, alkyloxythiocarbonyl, aryloxythiocarbonyl, alkyloxyalkylthiocarbonyl, aryloxyalkylthiocarbonyl, Het¹alkylthiocarbonyl, Het¹oxythiocarbonyl, Het¹alkyloxythiocarbonyl, alkylaminocarbonyl, alkenylaminocarbonyl, alkynylaminocarbonyl, arylaminocarbonyl, alkyloxyalkylaminocarbonyl, aryloxyalkylaminocarbonyl, cycloalkylaminocarbonyl, arylalkylaminocarbonyl, Het¹aminocarbonyl, Het¹alkylaminocarbonyl, Het¹oxyalkylaminocarbonyl, Het¹alkyloxyaminocarbonyl, alkylaminothiocarbonyl, alkenylaminothiocarbonyl, alkynylaminothiocarbonyl, arylaminothiocarbonyl, arylalkylaminothiocarbonyl, alkyloxyalkylaminothiocarbonyl, aryloxyalkylaminothiocarbonyl, Het¹alkylaminothiocarbonyl, Het¹aminothiocarbonyl, Het¹oxyalkylaminothiocarbonyl, Het¹alkyloxyalkylaminothiocarbonyl, Het¹aminoalkylpolyalkylamino, arylaminopolyalkylamino polyaminoalkyl, aminoarylpolyaminoalkyl and aminoalkyloxypolyaminoalkyl, wherein one or more carbon atoms of said radical are optionally substituted by one or more substituents independently selected from the group consisting of oxo, alkyl, aralkyl, aryl, Het¹, Het², cycloalkyl, alkyloxycarbonyl, carboxyl, aminocarbonyl, mono- or di(alkyl)amino-carbonyl, aminosulfonyl, alkylS(═O)ₜ, hydroxy, cyano, halogen, haloalkyl,-alkoxy, haloalkoxy, nitro, amino, mono- and disubstituted amino wherein the substituent(s) of said amino group is (are) independently selected from the group consisting of alkyl, aryl, arylalkyl, aryloxy, arylamino, arylthio, aryloxyalkyl, arylaminoalkyl, arylalkoxy, alkylthio, alkoxy, aryloxyalkoxy, arylaminoalkoxy, arylalkylamino, aryloxyalkylamino, arylaminoarylamino, alkylaminoarylamino, arylaminoalkylamino, arylthioalkoxy, arylthioalkylamino, arylalkylthio, aryloxy-alkylthio, arylaminoalkylthio, arylthioalkylthio, alkylamino, cycloalkyl, cycloalkyl-alkyl, Het¹, Het², Het¹alkyl, Het²alkyl, Het¹amino, Het²amino, Het¹alkylamino, Het²alkylamino, Het¹thio, Het²thio, Het¹alkylthio, Het²alkylthio, Het¹oxy, Het²oxy, OR¹¹, SR¹¹, SO₂NR¹¹R¹², SO₂₂N(OH)R¹¹, CN, CR¹¹═NR¹², S(O)R¹¹, SO₂R¹¹, CR¹¹═N(OR¹²), N₃, NO₂, NR¹¹R¹², N(OH)R¹¹, C(O)R¹¹, C(S)R¹¹, CO₂R¹¹, C(O)SR¹¹, C(O)NR¹¹R ², C(S)NR¹¹R¹², C(O)N(OH)R¹², C(S)N(OH)R¹¹, NR¹¹C(O)R¹², NR¹¹C(S)R¹², N(OH)C(O)R¹², N(OH)C(S)R¹¹, NR¹¹CO₂R¹², NR¹¹C(O)NR¹²R¹³, and NR¹¹C(S)NR¹²R¹³, N(OH)CO₂R¹¹, NR¹¹C(O)SR¹², N(OH)C(O)NR¹¹R¹², N(OH)C(S)NR¹¹R¹², NR¹¹C(O)N(OH)R¹², NR¹¹C(S)N(OH)R¹², NR¹¹SO₂R¹², NHSO₂NR¹¹R¹², NR¹¹SO₂NHR¹² and P(O)(OR¹¹)(OR¹²), wherein t is 1 or 2, and wherein R¹¹, R¹² and R¹³ are each independently selected from the group consisting of hydrogen, alkyl, alkenyl, and alkynyl;
   with the proviso that R₁ is not naphthalimido-alkylaminoalkyl when R′ is C₂₋₇ alkylcarbonyl, arylcarbonyl or cycloalkylcarbonyl; or by the general formula (II) 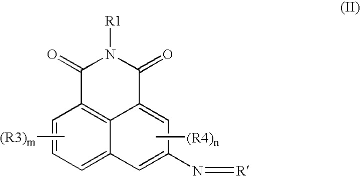
   wherein:
m, n, R₁, R₃ and R₄ are as defined with respect to formula (I), and
R′ is a radical selected from the group consisting of alkylidene, alkenylidene, arylalkenylidene, cycloalkylidene, cycloalkylalkylidene, arylalkylidene, Het¹ylidene, Het¹alkylidene, Het²alkylidene, alkylcarbonylalkylidene, alkenylcarbonylalkylidene, alkynylcarbonylalkylidene, arylcarbonylalkylidene, alkyloxycarbonylalkylidene, aryloxycarbonylalkylidene, aryloxyalkylcarbonylalkylidene, cycloalkylcarbonylalkylidene, arylalkylcarbonylalkylidene, Het¹carbonylalkylidene, Het¹alkylcarbonylalkylidene, Het¹ oxycarbonylalkylidene, Het¹alkyloxycarbonylalkylidene, alkylthiocarbonylalkylidene, alkenylthiocarbonylalkylidene, alkynylthiocarbonylalkylidene, arylthiocarbonylalkylidene, arylalkylthiocarbonylalkylidene, alkyloxythiocarbonylalkylidene, aryloxythiocarbonylalkylidene, alkyloxyalkylthiocarbonylalkylidene, aryloxyalkylthiocarbonylalkylidene, Het¹carbonylalkylidene, Het¹alkylthiocarbonylalkylidene, Het¹oxythiocarbonylalkylidene, Het¹alkyloxythiocarbonylalkylidene, alkylureylalkylidene, alkenylthioureylalkylidene, alkynylthioureylalkylidene, arylureylalkylidene, alkyloxyalkylureylalkylidene, aryloxyalkylureylalkylidene, cycloalkylureylalkylidene, arylalkylureylalkylidene, Het¹ureylalkylidene, Het¹alkylureylalkylidene, Het¹oxyalkylureylalkylidene, Het¹alkyloxyalkylureylalkylidene and alkylthioureylalkylidene, wherein one or more carbon atoms of said radical are optionally substituted by one or more substituents independently selected from the group consisting of alkyl, aralkyl, aryl, Het¹, Het², cycloalkyl, alkyloxycarbonyl, carboxyl, aminocarbonyl, mono- or di(alkyl)aminocarbonyl, aminosulfonyl, alkylS(═O)t, hydroxy, cyano, halogen, amino, mono- and disubstituted amino wherein the substituent(s) of said amino group is (are) independently selected from the group consisting of alkyl, aryl, arylalkyl, aryloxy, arylamino, arylthio, aryloxyalkyl, arylaminoalkyl, arylalkoxy, alkylthio, alkoxy, aryloxyalkoxy, arylaminoalkoxy, arylalkylamino, aryloxyalkylamino, arylaminoalkylamino, arylthioalkoxy, arylthioalkylamino, arylalkylthio, aryloxyalkylthio, arylaminoalkylthio, arylthioalkylthio, alkylamino, cycloalkyl, cycloalkylalkyl, Het¹, Het², Het¹alkyl, Het²alkyl, Het¹amino, Het²amino, Het¹alkylamino, Het²alkylamino, Het¹thio, Het²thio, Het¹alkylthio, Het²alkylthio, Het¹oxy, Het²oxy, OR¹¹, SR¹¹, SO₂NR¹¹R¹², SO₂N(OH)R¹¹, CN, CR¹¹═NR¹², S(O)R¹¹, SO₂R¹¹, CR¹¹═N(OR), N₃, NO₂, NR¹¹R¹², N(OH)R¹¹, C(O)R¹¹, C(S)R¹¹, CO₂R¹¹, C(O)SR¹¹, C(O)NR¹¹R¹², C(S)NR¹¹R¹², C(O)N(OH)R¹², C(S)N(OH)R¹¹, NR¹¹C(O)R¹², NR¹¹C(S)R¹², N(OH)C(O)R¹², N(OH)C(S)R¹¹, NR¹¹CO₂R¹², NR¹¹C(O)NR¹²R¹³, and NR¹¹C(S)NR¹²R¹³, N(OH)CO₂R¹¹, NR¹¹C(O)SR¹², N(OH)C(O)NR¹¹R¹², N(OH)C(S)NR¹¹R¹², NR¹¹C(O)N(OH)R¹², NR¹¹C(S)N(OH)R¹², NR¹¹SO₂R¹², NHSO₂NR¹¹R¹², NR¹¹SO₂NHR¹² and P(O)(OR¹¹)(OR¹²), wherein t is 1 or 2, and wherein R¹¹, R¹² and R¹³ are each independently selected from the group consisting of hydrogen, alkyl, alkenyl, and alkynyl;
   and/or a pharmaceutically acceptable salt thereof and/or a solvate thereof.

The above defined novel compounds have in common the structural feature that the amino group of an amino-substituted naphthalimide (isoquinolinedione) such as amonafide is substituted by a functional group, the term functional including both the presence of a carbonyl or thiocarbonyl or secondary amino group (formula I) and the presence of an imino unsaturation (formula II).

In a second embodiment, the invention provides a method for the production of substituted naphthalimide (isoquinolinedione) derivatives represented by the general formula (I) by reacting amonafide or an amonafide derivative with a reagent selected from the group consisting of acyl halides, thioacyl halides, isocyanates, isothiocyanates and polyamines. In a third embodiment, the invention provides a method for the production of substituted naphthalimide (isoquinolinedione) derivatives represented by the general formula (II) by reacting amonafide or an amonafide derivative with an aldehyde. In another embodiment, the invention provides a method for the production of addition salts and/or solvates of said substituted naphthalimide (isoquinolinedione) derivatives.

In another embodiment, the invention provides a pharmaceutical composition comprising:
a therapeutically effective amount of a substituted naphthalimide (isoquinolinedione) derivative represented by the general formula (I) or the general formula (II), and/or a pharmaceutically acceptable salt thereof and/or a solvate thereof and
one or more pharmaceutically acceptable carriers.

In another embodiment, the invention provides combined preparations containing at least one substituted naphthalimide (isoquinolinedione) derivative represented by the general formula (I) or the general formula (II) and/or a pharmaceutically acceptable salt thereof and/or a solvate thereof, and one or more antineoplastic drugs, preferably in the form of synergistic combinations as detailed below.

In another embodiment, the invention relates to the unexpected finding that substituted naphthalimide (isoquinolinedione) derivatives represented by the general formula (I) or the general formula (II), and/or a pharmaceutically acceptable salt thereof and/or a solvate thereof, have significantly higher biological activity, especially with respect to tumour cells, than amonafide while avoiding many of the above-mentioned drawbacks of amonafide. In particular, the naphthalimide derivatives according to the invention have a significant anti-migratory effect. Migration refers to the process whereby cells migrate from a neoplastic tumor tissue and colonize new tissues, using blood or lymphatic vessels as major routes of migration, this process being also known as the metastatic process. Based on this finding, the present invention provides a method for treating and/or preventing tumours in humans. More specifically, the invention relates to a method of treatment of a host with a cellular proliferative disease, comprising contracting said host with an effective amount of a substituted naphthalimide (isoquinolinedione) derivative represented by the general formula (I) or the general formula (II), and/or a pharmaceutically acceptable salt thereof and/or a solvate thereof.

In another embodiment, the invention provides the use of substituted naphthalimide (isoquinolinedione) derivatives represented by the general formula (I) or the general formula (II), and/or a pharmaceutically acceptable salt thereof and/or a solvate thereof, as anti-tumour agents agents.

### BRIEF DESCRIPTION OF THE DRAWINGS

FIG. 1 schematically shows a synthetic route for making a particular naphthalimide derivative according to the present invention.

FIG. 2 schematically shows a synthetic route for making another particular naphthalimide derivative according to the present invention.

### DEFINITIONS

As used herein with respect to a substituting radical, and unless otherwise stated, the term “alkyl” means straight and branched chain saturated acyclic hydrocarbon monovalent radicals having from 1 to 7 carbon atoms such as, for example, methyl, ethyl, propyl, n-butyl, 1-methylethyl (isopropyl), 2-methylpropyl (isobutyl), 1,1-dimethylethyl (ter-butyl), 2-methylbutyl, n-pentyl, dimethylpropyl, n-hexyl, 2-methylpentyl, 3-methylpentyl, n-heptyl, 2-methylhexyl and the like. When a narrower definition is intended, a notation such as C₂₋₇ alkyl is used, meaning that the radical has from 2 to 7 carbon atoms.

As used herein with respect to a substituting radical, and unless otherwise stated, the term “alkylene” means a divalent hydrocarbon radical corresponding to the above defined alkyl, such as but not limited to methylene, bis(methylene), tris(methylene), tetramethylene, hexamethylene and the like.

As used herein with respect to a substituting radical, and unless otherwise stated, the term “alkylidene” means a divalent hydrocarbon radical formally derived by removal of two hydrogen atoms from the same carbon atom of the corresponding alkyl, such as but not limited to methylidene, ethylidene and the like.

As used herein with respect to a substituting radical, and unless otherwise stated, the term “alkenyl” designates a straight and branched acyclic hydrocarbon monovalent radical having one or more ethylenic unsaturations and having from 2 to 7 carbon atoms such as, for example, vinyl, 1-propenyl, 2-propenyl (allyl), 1-butenyl, 2-butenyl, 2-pentenyl, 3-pentenyl, 3-methyl-2-butenyl, 3-hexenyl, 2-hexenyl, 2-heptenyl, 1,3-butadienyl, pentadienyl, hexadienyl, heptadienyl, heptatrienyl and the like, including all possible isomers thereof.

As used herein with respect to a substituting radical, and unless otherwise stated, the term “alkynyl” defines straight and branched chain hydrocarbon radicals containing one or more triple bonds and optionally at least one double bond and having from 2 to 7 carbon atoms such as, for example, acetylenyl, 1-propynyl, 2-propynyl, 1-butynyl, 2-butynyl, 2-pentynyl, 1-pentynyl, 3-methyl-2-butynyl, 3-hexynyl, 2-hexynyl, 1-penten-4-ynyl, 3-penten-1-ynyl, 1,3-hexadien-1-ynyl and the like.

As used herein with respect to a substituting radical, and unless otherwise stated, the term “cycloalkyl” means a mono- or polycyclic saturated hydrocarbon monovalent radical having from 3 to 10 carbon atoms, such as for instance cyclopropyl, cyclobutyl, cyclopentyl, cyclohexyl, cycloheptyl, cyclooctyl and the like, or a C₇₋₁₀ polycyclic saturated hydrocarbon monovalent radical having from 7 to 10 carbon atoms such as, for instance, norbornyl, fenchyl, trimethyltricycloheptyl or adamantyl.

As used herein with respect to a substituting radical, and unless otherwise stated, the term “cycloalkylene” means the divalent hydrocarbon radical corresponding to the above defined cycloalkyl.

As used herein with respect to a substituting radical, and unless otherwise stated, the term “acyl” broadly refers to a carbonyl (oxo) group adjacent to an alkyl radical, a cycloalkyl radical, an aryl radical, an arylalkyl radical or a heterocyclic (including Het¹ and Het²) radical, all of them being such as herein defined; representative examples include acetyl, benzoyl, naphthoyl and the like; similarly, the term “thioacyl” refers to a C═S (thioxo) group adjacent to one of the said radicals.

As used herein with respect to a substituting radical, and unless otherwise stated, the term “cycloalkylalkyl” refers to an aliphatic saturated hydrocarbon monovalent radical (preferably an alkyl such as defined above) to which a cycloalkyl (such as defined above) is already linked such as, but not limited to, cyclohexylmethyl, cyclopentylmethyl and the like.

As used herein with respect to a substituting radical, and unless otherwise stated, the term “aryl” designate any mono- or polycyclic aromatic monovalent hydrocarbon radical having from 6 to 30 carbon atoms such as but not limited to phenyl, naphthyl, anthracenyl, phenantracyl, fluoranthenyl, chrysenyl, pyrenyl, biphenylyl, terphenyl, picenyl, indenyl, biphenyl, indacenyl, benzocyclobutenyl, benzocyclooctenyl and the like, including fused benzoC₄₋₈ cycloalkyl radicals (the latter being as defined above) such as, for instance, indanyl, tetrahydronaphtyl, fluorenyl and the like, each of said radicals being optionally substituted with one or more substituents independently selected from the group consisting of halogen, amino, cyano, trifluoromethyl, hydroxyl, sulfhydryl and nitro, such as for instance 4-fluorophenyl, 4-chlorophenyl, 3,4-dichlorophenyl, 4-cyanophenyl, 2,6-dichlorophenyl, 2-fluorophenyl, 3-chlorophenyl, 3,5-dichlorophenyl and the like.

As used herein with respect to a substituting radical, and unless otherwise stated, the term “arylene” means a divalent hydrocarbon radical corresponding to the above defined aryl, such as but not limited to phenylene, naphthylene, indenylidene and the like.

As used herein with respect to a substituting radical, and unless otherwise stated, the term “Het¹” alone or in combination with another radical is defined as a saturated or partially unsaturated monocyclic, bicyclic or polycyclic heterocycle having preferably 3 to 12 ring members, more preferably 5 to 10 ring members and more preferably 5 to 6 ring members, which contains one or more heteroatom ring members selected from the group consisting of nitrogen, oxygen or sulfur and wherein one or more carbon atoms of said heterocycle is optionally substituted by one or more substituents selected from the group consisting of alkyl, alkoxy, halogen, hydroxy, oxo, sulhydryl, thioxo, thioalkyl, amino, nitro, cyano, haloalkyl, carboxyl, alkoxycarbonyl, cycloalkyl, aminocarbonyl, methylthio, methylsulfonyl, aryl, saturated or partially unsaturated monocyclic, bicyclic and tricyclic heterocycles having 3 to 12 ring members and having one or more heteroatom ring members selected from the group consisting of nitrogen, oxygen and sulfur, and mono- and disubstituted amino, and mono- and disubstituted aminocarbonyl, whereby the optional substituents on any amino function are independently selected from the group consisting of alkyl, alkoxy, Het², Het²alkyl, Het²oxy, Het²oxyalkyl, aryl, aryloxy, aryloxyalkyl, arylalkyl, alkyloxycarbonylamino, amino and aminoalkyl, wherein each of the latter amino groups may optionally be mono- or where possible di-substituted with alkyl;

As used herein with respect to a substituting radical, and unless otherwise stated, the term “Het²” alone or in combination with another radical is defined as an aromatic monocyclic, bicyclic or tricyclic heterocycle having preferably 3 to 12 ring members, more preferably 5 to 10 ring members and more preferably 5 to 6 ring members, which contains one or more heteroatom ring members selected from the group consisting of nitrogen, oxygen or sulfur and wherein one or more carbon atoms of said heterocycle is optionally substituted by one or more substituents selected from the group consisting of alkyl, alkoxy, halogen, hydroxy, oxo, sulhydryl, thioxo, thioalkyl, amino, nitro, cyano, haloalkyl, carboxyl, alkoxycarbonyl, cycloalkyl, aminocarbonyl, methylthio, methylsulfonyl, aryl, Het¹ and monocyclic, bicyclic or tricyclic heterocycles having 3 to 12 ring members and having one or more heteroatom ring members selected from the group consisting of nitrogen, oxygen and sulfur, and mono- and disubstituted amino, and mono- and disubstituted aminocarbonyl, whereby the optional substituents on any amino function are independently selected from the group consisting of alkyl, alkoxy, Het¹, Het¹alkyl, Het¹oxy, Het¹oxyalkyl, aryl, aryloxy, aryloxyalkyl, arylalkyl, alkyloxycarbonylamino, amino and aminoalkyl, wherein each of the lafter amino groups may optionally be mono- or where possible di-substituted with alkyl;

As used herein with respect to a substituting radical, and unless otherwise stated, the term “heterocyclic” includes both Het¹ and Het²; specific examples thereof include, but are not limited to, naphthalimidyl, diazepinyl, oxadiazinyl, thiadiazinyl, dithiazinyl, triazolonyl, diazepinonyl, triazepinyl, triazepinonyl, tetrazepinonyl, benzoquinolinyl, benzothiazinyl, benzothiazi-nonyl, benzoxathiinyl, benzodioxinyl, benzodithiinyl, benzoxazepinyl, benzothiazepinyl, benzodiazepinyl, benzodioxepinyl, benzodithiepinyl, benzoxazocinyl, benzothiazocinyl, benzodiazocinyl, benzoxathiocinyl, benzodioxocinyl, benzotrioxepinyl, benzoxathiazepinyl, benzoxadiazepinyl, benzothiadiazepinyl, benzotriazepinyl, benzoxathiepinyl, benzotriazinonyl, benzoxazolinonyl, azetidinonyl, azaspiroundecyl, dithiaspirodecyl, hypoxanthinyl, azahypoxanthinyl, bipyrazinyl, bipyridinyl, oxazolidinyl, benzodioxocinyl, benzopyrenyl, benzopyranonyl, benzophenazinyl, benzoquinolizinyl, dibenzocarbazolyl, dibenzoacridinyl, dibenzophenazinyl, dibenzothiepinyl, dibenzooxepinyl, dibenzopyranonyl, dibenzoquinoxalinyl, dibenzothiazepinyl, dibenzoisoquinolinyl, tetraazaadamantyl, thiatetraaza-adamantyl, oxauracil, oxazinyl, dibenzothiophenyl, dibenzofuranyl, oxazolinyl, oxazolonyl, azaindolyl, azolonyl, thiazolinyl, thiazolonyl, thiazolidinyl, thiazanyl, pyrimidonyl, thiopyrimidonyl, thiamorpholinyl, azlactonyl, naphtindazolyl, naphtindolyl, naphtothiazolyl, naphtothioxolyl, naphtoxindolyl, naphtotriazolyl, naphtopyranyl, oxabicycloheptyl, azabenzimidazolyl, azacycloheptyl, azacyclooctyl, azacyclononyl, azabicyclononyl, tetrahydrofuryl, tetrahydro-pyranyl, tetrahydropyronyl, tetrahydroquinoleinyl, tetrahydrothienyl and dioxide thereof, dihydrothienyl dioxide, dioxindolyl, dioxinyl, dioxenyl, dioxazinyl, thioxanyl, thioxolyl, thiourazolyl, thiotriazolyi, thiopyranyl, thiopyronyl, coumarinyl, quinoleinyl, oxyquinoleinyl, quinuclidinyl, xanthinyl, dihydropyranyl, benzodihydrofuryl, benzothiopyronyl, benzothiopyranyl, benzoxazinyl, benzoxazolyl, benzodioxolyl, benzodioxanyl, benzothiadiazolyl, benzotriazinyl, benzothiazolyl, benzoxazolyl, phenothioxinyl, phenothiazolyl, phenothienyl (benzothiofuranyl), phenopyronyl, phenoxazolyl, pyridinyl, dihydropyridinyl, tetrahydropyridinyl, piperidinyl, morpholinyl, thiomorpholinyl, pyrazinyl, pyrimidinyl, pyridazinyl, triazinyl, tetrazinyl, triazolyl, benzotriazolyl, tetrazolyl, imidazolyl, pyrazolyl, thiazolyl, thiadiazolyl, isothiazolyl, oxazolyl, oxadiazolyl, pyrrolyl, furyl, dihydrofuryl, furoyl, hydantoinyl, dioxolanyl, dioxolyl, dithianyl, dithienyl, dithiinyl, thienyl, indolyl, indazolyl, benzofuryl, quinolyl, quinazolinyl, quinoxalinyl, carbazolyl, phenoxazinyl, phenothiazinyl, xanthenyl, purinyl, benzothienyl, naphtothienyl, thianthrenyl, pyranyl, pyronyl, benzopyronyl, isobenzofuranyl, chromenyl, phenoxathiinyl, indolizinyl, quinolizinyl, isoquinolyl, phthalazinyl, naphthiridinyl, cinnolinyl, pteridinyl, carbolinyl, acridinyl, perimidinyl, phenanthrolinyl, phenazinyl, phenothiazinyl, imidazolinyl, imidazolidinyl, benzimidazolyl, pyrazolinyl, pyrazolidinyl, pyrrolinyl, pyrrolidinyl, piperazinyl, uridinyl, thymidinyl, cytidinyl, azirinyl, aziridinyl, diazirinyl, diaziridinyl, oxiranyl, oxaziridinyl, dioxiranyl, thiiranyl, azetyl, dihydroazetyl, azetidinyl, oxetyl, oxetanyl, oxetanonyl, thietyl, thietanyl, diazabicyclooctyl, diazetyl, diaziridinonyl, diaziridinethionyl, chromanyl, chromanonyl, thiochro-manyl, thiochromanonyl, thiochromenyl, benzofuranyl, benzisothiazolyl, benzocarbazolyl, benzochromonyl, benzisoalloxazinyl, benzocoumarinyl, thiocoumarinyl, phenometoxazinyl, phenoparoxazinyl, phentriazinyl, thio-diazinyl, thiodiazolyl, indoxyl, thioindoxyl, benzodiazinyl (e.g. phtalazinyl), phtalidyl, phtalimidinyl, phtalazonyl, alloxazinyl, dibenzo-pyronyl (i.e. xanthonyl), xanthionyl, isatyl, isopyrazolyl, isopyrazolonyl, urazolyl, urazinyl, uretinyl, uretidinyl, succinyl, succinimido, benzylsultimyl, benzylsultamyl and the like, including all possible isomeric forms thereof.

As used herein with respect to a substituting radical, and unless otherwise stated, the term “Het¹-ylidene” means a divalent radical formally derived by removal of two hydrogen atoms from the same carbon atom of the corresponding Het¹ radical, such as but not limited to pyrrolinylidene, piperidinylidene and the like.

As used herein with respect to a substituting radical, and unless otherwise stated, the terms “alkoxy”, “aryloxy”, “arylalkyloxy”, “thioalkyl”, “arylthio” and “arylalkylthio” refer to substituents wherein an alkyl radical, respectively an aryl or arylalkyl radical (each of them such as defined herein), are attached to an oxygen atom or a divalent sulfur atom through a single bond, such as but not limited to methoxy, ethoxy, propoxy, butoxy, pentoxy, isopropoxy, sec-butoxy, tert-butoxy, isopentoxy, cyclopropyloxy, cyclobutyl-oxy, cyclopentyloxy, thiomethyl, thioethyl, thiopropyl, thiobutyl, thiopentyl, thiocyclopropyl, thiocyclobutyl, thiocyclopentyl, thiophenyl, phenyloxy, benzyloxy, mercapto-benzyl, cresoxy and the like.

As used herein with respect to a substituting atom, and unless otherwise stated, the term “halogen” means any atom selected from the group consisting of fluorine, chlorine, bromine and iodine.

As used herein with respect to a substituting radical, and unless otherwise stated, the term “haloalkyl” means an alkyl radical (such as above defined) in which one or more hydrogen atoms are independently replaced by one or more halogens (preferably fluorine, chlorine or bromine), such as but not limited to difluoromethyl, trifluoromethyl, trifluoroethyl, octafluoropentyl, dodecafluoroheptyl, dichloromethyl and the like.

As used herein with respect to a substituting radical, and unless otherwise stated, the terms “arylalkyl”, “cycloalkylalkyl”, “Het¹alkyl” and “Het²alkyl” refer to an aliphatic saturated hydrocarbon monovalent radical (preferably an alkyl radical such as defined above) onto which an aryl, cycloalkyl, Het¹ or Het² radical (such as defined above) is already linked, and wherein the said aliphatic radical and/or the said aryl or Het¹ or Het² radical may be optionally substituted with one or more substituents for instance independently selected from the group consisting of C₁₋₄ alkyl, trifluoromethyl, halogen, amino, nitro, hydroxyl, sulfhydryl and nitro, such as but not limited to benzyl, 4-chlorobenzyl, 2-fluorobenzyl, 4-fluorobenzyl, 3,4-dichlorobenzyl, 2,6-dichlorobenzyl, 4-ter-butylbenzyl, 3-methylbenzyl, 4-methylbenzyl, phenylpropyl, 1-naphtylmethyl, phenylethyl, 1-amino-2-phenyl-ethyl, 1-amino-2-[4-hydroxyphenyl]ethyl, 1-amino-2-[indol-2-yl]ethyl, styryl, pyridylmethyl (including all isomers thereof), pyridylethyl, 2-(2-pyridyl)-isopropyl, oxazolylbutyl, 2-thienylmethyl, pyrrolyiethyl, morpholinylethyl, imidazoi-1-yl-ethyl, benzodioxolylmethyl, cyclohexylmethyl, cyclopentylmethyl and 2-furylmethyl.

As used herein with respect to a substituting radical, and unless otherwise stated, the term “arylalkylidene” refers to an aliphatic divalent radical (preferably an alkylidene radical such as defined above) onto which one or two aryl radicals (such as defined above) is (are) already linked, such as but not limited to benzylidene, diphenylmethylene and the like.

As used herein with respect to a substituting radical, and unless otherwise stated, the term “protected amino” refers to an amino group being protected by N-protecting groups including acyl groups such as formyl, acetyl, propionyl, pivaloyl, tert-butylacetyl, 2-chloroacetyl, 2-bromoacetyl, trifluoracetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl, and the like, carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 4-methoxybenzyloxycarbonyl, 2-vitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, tert-butoxycarbonyl, diisopropylmethoxycarbonyl, isopropyloxycarbonyl, ethoxycarbonyl, methoxycarbonyl, allyloxycarbonyl, 2,2,2-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl, and the like; alkyl groups such as benzyl, triphenylmethyl, benzyloxymethyl and the like; and silyl groups such as trimethylsilyl and the like.

As used herein with respect to a substituting radical, and unless otherwise stated, the term “monoalkylamino” means that a single C₁₋₇ alkyl radical such as defined herein is attached to a nitrogen atom through a single bond such as, but not limited to, methylamino, ethylamino, isopropylamino, n-butylamino, and tert-butylamino.

As used herein with respect to a substituting radical, and unless otherwise stated, the term “dialkylamino” means that two C₁₋₇ alkyl radicals independently defined as specified herein are each attached to the same nitrogen atom through a single bond such as, but not limited to, dimethylamino, diethylamino, diisopropylamino, di-n-butylamino, di-tert-butylamino, and ethylmethylamino.

As used herein and unless otherwise stated, the term “solvate” includes any combination which may be formed by a naphthalimide (isoquinolinedione) or 1,2-dihydro-3H-dibenzisoquino-line-1,3-dione derivative of this invention with a suitable inorganic solvent (e.g. hydrates) or organic solvent, such as but not limited to alcohols, ketones, esters and the like.

As used herein and unless otherwise stated, the term “anti-migratory” refers to the ability of a pharmaceutical ingredient to stop the migration of cells away from the neoplastic tumor tissue and thus to reduce the colonization of new tissues by these cells.

### DETAILED DESCRIPTION OF THE INVENTION

In a first aspect, the invention provides a family of substituted naphthalimide (isoquinolinedione) derivatives represented by the general formula (I) 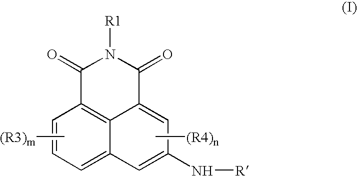
wherein each of m, n, R₁, R₃, R′ and R₄ are as broadly defined hereinabove, and/or a pharmaceutically acceptable salt thereof and/or a solvate thereof. Within this broad family, the following embodiments are preferred:
n=0, and/or
m=0, and/or
m=2, both substituents R₃ being adjacent and together with the carbon atoms to which they are attached forming a phenyl group, and/or
R₁ is an alkylene radical having from 1 to 3 carbon atoms and linked to a nitrogen-containing group selected from the group consisting of dimethylamino, diethylamino, pyrrolidino, piperidino, N-methylpiperazino, morpholino and ureyl, more preferably R₁ is dimethylene linked to dimethylamino or diethylamino, and/or
R′ is selected from the group consisting of C₂₋₇ alkylcarbonyl, amino-carbonyl, thioaminocarbonyl, alkylaminocarbonyl, alkylthioaminocarbonyl, alkylthiocarbonyl and poly(aminoalkyl) wherein the number of aminoalkyl repeating units is within a range from 2 to about 5.

In a second aspect, the invention provides a family of substituted naphthalimide (isoquinolinedione) derivatives represented by the following general formula (II) 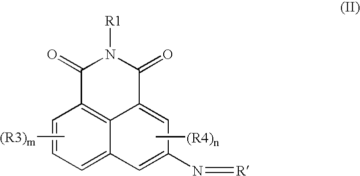
wherein each of m, n, R₁, R₃, R′ and R₄ are as broadly defined hereinabove, and/or a pharmaceutically acceptable salt thereof and/or a solvate thereof. Within this broad family, the following embodiments are preferred:
n=0, and/or
m=0, and/or
m=2, both substituents R₃ being adjacent and together with the carbon atoms to which they are attached forming a phenyl group, and/or
R₁ is an alkylene radical having from 1 to 3 carbon atoms and linked to a nitrogen-containing group selected from the group consisting of dimethylamino, diethylamino, pyrrolidino, piperidino, N-methylpiperazino, morpholino and ureyl, more preferably R₁ is dimethylene linked to dimethylamino or diethylamino, and/or
R′ is selected from the group consisting of arylalkylidene (e.g. benzylidene and substituted derivatives thereof, Het¹-ylidene (e.g. pyrrolinylidene), Het¹alkylidene, alkylidene (such as, but not limited to, methylidene, ethylidene and n-propylidene) and cycloalkylidene (e.g. cyclohexylidene and norbornylidene).

In another particular embodiment, the invention relates to a group of naphthalimide (isoquinolinedione) derivatives, as well as pharmaceutical compositions comprising such naphthalimide (isoquinolinedione) derivatives as active principle, having the above general formulae (I) or (II) and being in the form of a pharmaceutically acceptable salt. The latter include any therapeutically active non-toxic salt which compounds having the general formulae (I) or (II) are able to form with a salt-forming agent. Such addition salts may conveniently be obtained by treating the naphthalimide (isoquinolinedione) derivatives of the invention with an appropriate salt-forming acid or base. For instance, naphthalimide (isoquinolinedione) derivatives having basic properties may be converted into the corresponding therapeutically active, non-toxic acid salt form by treating the free base form with a suitable amount of an appropiate acid following conventional procedures. Examples of such appropriate salt-forming acids include, for instance, inorganic acids resulting in forming salts such as but not limited to hydrohalides (e.g. hydrochloride and hydrobromide), sulfate, nitrate, phosphate, diphosphate, carbonate, bicarbonate, and the like; and organic monocarboxylic or dicarboxylic acids resulting in forming salts such as, for example, acetate, propanoate, hydroxyacetate, 2-hydroxypropanoate, 2-oxopropanoate, lactate, pyruvate, oxalate, malonate, succinate, maleate, fumarate, malate, tartrate, citrate, methanesulfonate, ethanesulfonate, benzoate, 2-hydroxybenzoate, 4-amino-2-hydroxybenzoate, benzene-sulfonate, p-toluene-sulfonate, salicylate, p-aminosalicylate, pamoate, bitartrate, camphorsulfonate, edetate, 1,2-ethanedisulfonate, fumarate, glucoheptonate, gluconate, glutamate, hexylresorcinate, hydroxynaphtoate, hydroxyethanesulfonate, mandelate, methylsulfate, pantothenate, stearate, as well as salts derived from ethanedioic, propanedioic, butanedioic, (Z)-2-butenedioic, (E)2-butenedioic, 2-hydroxybutanedioic, 2,3-dihydroxybutane-dioic, 2-hydroxy-1,2,3-propane-tricarboxylic, cyclohexane-sulfamic acid and the like.

Naphthalimide (isoquinolinedione) derivatives having the general formulae (I) or (II) having acidic properties may be converted in a similar manner into the corresponding therapeutically active, non-toxic base salt form. Examples of appropriate salt-forming bases include, for instance, inorganic bases like metallic hydroxides such as but not limited to those of alkali and alkaline-earth metals like calcium, lithium, magnesium, potassium and sodium, or zinc, resulting in the corresponding metal salt; organic bases such as but not limited to ammonia, alkylamines, benzathine, hydrabamine, arginine, lysine, N,N′-dibenzyl-ethylenediamine, chloroprocaine, choline, diethanolamine, ethylenediamine, N-methylglucamine, procaine and the like.

Reaction conditions for treating the naphthalimide (isoquinolinedione) derivatives (I) or (II) of this invention with an appropriate salt-forming acid or base are similar to standard conditions involving the same acid or base but different organic compounds with basic or acidic properties, respectively. Preferably, in view of its use in a pharmaceutical composition or in the manufacture of medicament for treating specific diseases, the pharmaceutically acceptable salt will be designed, i.e. the salt-forming acid or base will be selected so as to impart greater water-solubility, lower toxicity, greater stability and/or slower dissolution rate to the naphthalimide (isoquinolinedione) derivative of this invention.

In another aspect the invention relates to methods for making substituted naphthalimide (isoquinolinedione) derivatives represented by the general formula wherein each of m, n, R₁, R₃, R′ and R₄ are as broadly defined hereinabove, by 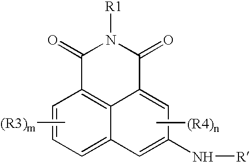
reacting amonafide (i.e. N-(2-dimethylaminoethyl)-3-amino-1,8-naphthalimide) or an amonafide derivative (i.e. a N-(R₁-substituted)-3-amino-1,8-naphthalimide optionally having m substituents R₃ and/or n substituents R₄) with an R′-containing reagent being able to react with the 3-amino group of amonafide or the amonafide (naphthalimide) derivative without substantially reacting with other substituents that may be present on the naphthalimide ring. Suitable examples of such reagents include the following:
R′-containing acyl halides or thioacyl halides, preferably R′-containing acyl chlorides or thioacyl chlorides wherein R′ is a radical selected from the group consisting of C₁₋₇ alkylcarbonyl, alkenylcarbonyl, alkynylcarbonyl, arylcarbonyl, aryloxycarbonyl, aryloxyalkylcarbonyl, cycloalkylcarbonyl, arylalkylcarbonyl, Het¹carbonyl, Het¹alkylcarbonyl, Het¹oxycarbonyl, Het¹alkyloxycarbonyl, alkylthiocarbonyl, alkenylthio-carbonyl, alkynylthiocarbonyl, arylthiocarbonyl, arylalkylthiocarbonyl, alkyloxythiocarbonyl, aryloxythiocarbonyl, alkyloxyalkylthiocarbonyl, aryloxyalkylthiocarbonyl, Het¹carbonyl, Het¹alkylthiocarbonyl, Het¹oxythiocarbonyl and Het¹alkyloxythiocarbonyl, wherein one or more carbon atoms of said radical are optionally substituted by one or more substituents independently selected from the group consisting of oxo, alkyl, aralkyl, aryl, Het¹, Het², cycloalkyl, alkyloxycarbonyl, carboxyl, aminocarbonyl, mono- or di(alkyl)aminocarbonyl, aminosulfonyl, alkylS(═O)ₜ, hydroxy, cyano, halogen, amino, mono- and disubstituted amino wherein the substituent(s) of said amino group is (are) independently selected from the group consisting of alkyl, aryl, arylalkyl, aryloxy, arylamino, arylthio, aryloxyalkyl, arylaminoalkyl, arylalkoxy, alkylthio, alkoxy, aryloxyalkoxy, arylaminoalkoxy, arylalkylamino, aryloxyalkylamino, arylaminoarylamino, alkylaminoarylamino, arylaminoalkylamino, arylthioalkoxy, arylthioalkylamino, arylalkylthio, aryloxyalkylthio, arylaminoalkylthio, arylthioalkylthio, alkylamino, cycloalkyl, cycloalkylalkyl, Het¹, Het², Het¹alkyl, Het²alkyl, Het¹amino, Het²amino, Het¹alkylamino, Het²alkylamino, Het¹thio, Het²thio, Het¹alkylthio, Het²alkylthio, Het¹oxy, Het²oxy, OR¹¹, SR¹¹, SO₂NR¹¹R¹², SO₂N(OH)R¹¹, CN, CR¹¹═NR¹², S(O)R¹¹, SO₂R¹¹, CR¹¹═N(OR¹²), N₃, NO₂, NR¹¹R¹², N(OH)R¹¹, C(O)R¹¹, C(S)R¹¹, CO₂R¹¹, C(O)SR¹¹, C(O)NR¹¹R¹², C(S)NR¹¹R¹², C(O)N(OH)R¹², C(S)N(OH)R¹¹, NR¹¹C(O)R¹², NR¹¹C(S)R¹², N(OH)C(O)R¹², N(OH)C(S)R¹¹, NR¹¹CO₂R¹², NR¹¹C(O)NR¹²R¹³, and NR¹¹C(S)NR¹²R¹³, N(OH)CO₂R¹¹, NR¹¹C(O)SR¹², N(OH)C(O)NR¹¹R¹², N(OH)C(S)NR¹¹R¹², NR¹¹C(O)N(OH)R¹², NR¹¹C(S)N(OH)R¹², NR¹¹SO₂R¹², NHSO₂NR¹¹R¹², NR¹¹SO₂NHR¹² and P(O)(OR¹¹)(OR¹²), wherein t is 1 or 2, and wherein R¹¹, R¹² and R¹³ are each independently selected from the group consisting of hydrogen, alkyl, alkenyl, and alkynyl;
R′-containing monoisocyanates and isothiocyanates wherein R′ is a radical selected from the group consisting of alkylaminocarbonyl, alkenylaminocarbonyl, alkynylaminocarbonyl, arylaminocarbonyl, alkyloxyaminocarbonyl, aryloxyaminocarbonyl, aryloxyalkylaminocarbonyl, cycloalkylaminocarbonyl, arylalkylaminocarbonyl, Het¹aminocarbonyl, Het¹alkylaminocarbonyl, Het¹oxyalkylaminocarbonyl, Het¹alkyloxyalkylaminocarbonyl, alkylthioaminocarbonyl, alkenylthioaminocarbonyl, alkynylthioaminocarbonyl, arylthioaminocarbonyl, arylalkylthioaminocarbonyl, alkyloxyalkylthioaminocarbonyl, aryloxyalkylthioaminocarbonyl, Het¹alkylthioaminocarbonyl, Het¹ oxyalkylthioaminocarbonyl, Het¹alkyloxyalkylthioaminocarbonyl, wherein one or more carbon atoms of said radical are optionally substituted by one or more substituents independently selected from the group consisting of oxo, alkyl, aralkyl, aryl, Het¹, Het², cycloalkyl, alkyloxycarbonyl, carboxyl, aminocarbonyl, mono- or di(alkyl)aminocarbonyl, aminosulfonyl, alkylS(═O)ₜ, hydroxy, cyano, halogen, amino, mono- and disubstituted amino wherein the substituent(s) of said amino group is (are) independently selected from the group consisting of alkyl, aryl, arylalkyl, aryloxy, arylamino, arylthio, aryloxyalkyl, arylaminoalkyl, arylalkoxy, alkylthio, alkoxy, aryloxyalkoxy, arylaminoalkoxy, arylalkylamino, aryloxyalkylamino, arylaminoarylamino, alkylaminoarylamino, arylaminoalkylamino, arylthioalkoxy, arylthioalkylamino, arylalkylthio, aryloxyalkylthio, arylaminoalkylthio, arylthioalkylthio, alkylamino, cycloalkyl, cycloalkylalkyl, Het¹, Het², Het¹alkyl, Het²alkyl, Het¹amino, Het²amino, Het¹alkylamino, Het²alkylamino, Het¹thio, Het²thio, Het¹alkylthio, Het²alkylthio, Het¹oxy, Het²oxy, OR¹¹, SR¹¹, SO₂NR¹¹R¹², SO₂N(OH)R¹¹, CN, CR¹¹═NR¹², S(O)R¹¹, SO₂R¹¹, CR¹¹═N(OR ¹²), N₃, NO₂, NR¹¹R¹², N(OH)R¹¹, C(O)R¹¹, C(S)R¹¹, CO₂R¹¹, C(O)SR¹¹, C(O)NR¹¹R¹², C(S)NR¹¹R¹², C(O)N(OH)R¹², C(S)N(OH)R¹¹, NR¹¹C(O)R¹², NR¹¹C(S)R¹², N(OH)C(O)R¹², N(OH)C(S)R¹¹, NR¹¹CO₂R¹², NR¹¹C(O)NR¹²R¹³, and NR¹¹C(S)NR¹²R¹³, N(OH)CO₂R¹¹, NR¹¹C(O)SR¹², N(OH)C(O)NR¹¹R¹², N(OH)C(S)NR¹¹R¹², NR¹¹C(O)N(OH)R¹², NR¹¹C(S)N(OH)R¹², NR¹¹SO₂R¹², NHSO₂NR¹¹R¹², NR¹¹SO₂NHR¹² and P(O)(OR¹¹)(OR¹²), wherein t is 1 or 2, and wherein R¹¹, R¹² and R¹³ are each independently selected from the group consisting of hydrogen, alkyl, alkenyl, and alkynyl;
R′-containing polyamines wherein R′ is a radical selected from the group consisting of Het¹aminoalkylpolyalkylamino, arylaminopolyalkylamino, polyaminoalkyl, aminoarylpolyaminoalkyl and aminoalkyloxypolyaminoalkyl, wherein one or more carbon atoms of said radical are optionally substituted by one or more substituents independently selected from the group consisting of oxo, alkyl, aralkyl, aryl, Het¹, Het², cycloalkyl, alkyloxycarbonyl, carboxyl, aminocarbonyl, mono- or di(alkyl)-aminocarbonyl, aminosulfonyl, alkylS(═O)ₜ, hydroxy, cyano, halogen, amino, mono- and disubstituted amino wherein the substituent(s) of said amino group is (are) independently selected from the group consisting of alkyl, aryl, arylalkyl, aryloxy, arylamino, arylthio, aryloxyalkyl, arylaminoalkyl, arylalkoxy, alkylthio, alkoxy, aryloxyalkoxy, arylamino-alkoxy, arylalkylamino, aryloxyalkylamino, arylaminoarylamino, alkylaminoarylamino, arylaminoalkylamino, arylthioalkoxy, arylthioalkylamino, arylalkylthio, aryloxyalkylthio, arylaminoalkylthio, arylthioalkylthio, alkylamino, cycloalkyl, cycloalkylalkyl, Het¹, Het², Het¹alkyl, Het²alkyl, Het¹amino, Het²amino, Het¹alkylamino, Het²alkylamino, Het¹thio, Het²thio, Het¹alkylthio, Het²alkylthio, Het¹oxy, Het²oxy, OR¹¹, SR¹¹, SO₂NR¹¹R¹², SO₂N(OH)R¹¹, CN, CR¹¹═NR¹², S(O)R¹¹, SO₂R¹¹, CR¹¹═N(OR¹²), N₃, NO₂, NR¹¹R¹², N(OH)R¹¹, C(O)R¹¹, C(S)R¹¹, CO₂R¹¹, C(O)SR¹¹, C(O)NR¹¹R¹², C(S)NR¹¹R¹², C(O)N(OH)R¹², C(S)N(OH)R¹¹, NR¹¹C(O)R¹², NR¹¹C(S)R¹², N(OH)C(O)R¹², N(OH)C(S)R¹¹, NR¹¹CO₂R¹², NR¹¹C(O)NR¹²R¹³, and NR¹¹C(S)NR¹²R¹³, N(OH)CO₂R¹¹, NR¹¹C(O)SR¹², N(OH)C(O)NR¹¹R¹², N(OH)C(S)NR¹¹R¹², NR¹¹C(O)N(OH)R¹², NR¹¹C(S)N(OH)R¹², NR¹¹SO₂R¹², NHSO₂NR¹¹R¹², NR¹¹SO₂NHR¹² and P(O)(OR¹¹)(OR¹²), wherein t is 1 or 2, and wherein R¹¹, R¹² and R¹³ are each independently selected from the group consisting of hydrogen, alkyl, alkenyl, and alkynyl.

Such reaction may be performed in any suitable solvent system for both reagents such as but not limited to acetonitrile or pyridine, or even in special circumstances by using said reagent as the solvent. Reaction may usually be effected at moderate temperatures (i.e. between about 15° C. and about 45° C.), although the reaction rate may be increased by heating up to the boiling temperature of the solvent. Reaction is preferably carried out by using an at least stoechiometric amount, more preferably a molar ratio in the range from about 1.1 to about 3.0, of the R′-containing reagent with respect to the isoquinolinedione derivative.

In another aspect the invention relates to a method of making a substituted naphthalimide (isoquinolinedione) derivatives represented by the general formula (II) 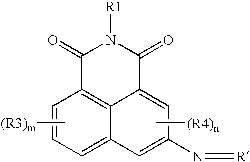
wherein each of m, n, R₁, R₃, R′ and R₄ are as broadly defined hereinabove, by reacting amonafide (i.e. N-(2-dimethylaminoethyl)-3-amino-1,8-naphthalimide) or an amonafide derivative (i.e. a N-(R₁-substituted)-3-amino-1,8-naphthalimide optionally having m substituents R₃ and/or n substituents R₄) with an aldehyde having the formula R′CH(O). Said aldehyde may be formaldehyde or may be aliphatic (e.g. acetaldehyde, propionaldehyde, butanaldehyde or valeraldehyde), ethylenically unsaturated aliphatic (e.g. allyl aldehyde and crotonaldehyde), saturated cycloaliphatic (e.g. cyclohexanecarboxaldehyde , cyclooctanecarboxaldehyde), ethylenically unsaturated cycloaliphatic (e.g. 3-cyclohexene-1-carboxaldehyde), arylalkenyl (e.g. cinnamaldehyde), aromatic (e.g. benzaldehyde and substituted derivatives thereof such as, but not limited to, salicylaidehyde, tolualdehyde, anisaldehyde, 2,5-dihydroxybenzaldehyde, 4-propoxybenzaldehyde, 4-phenoxybenzaldehyde, 3-(3,4-dichlorophenoxy)benzaldehyde, 3-(3,5-dichlorophenoxy) benzaldehyde, 2-bromobenzaldehyde, 3-bromobenzaldehyde, 4-bromo-benzaldehyde, 2-chlorobenzaldehyde, 3-chlorobenzaldehyde, 4-chlorobenzaldehyde, 2-fluorobenzaldehyde, 3-fluorobenzaldehyde, 4-fluorobenzaldehyde, 2,3-dichlorobenzaldehyde, 2,4-dichlorobenzaldehyde, 2,6-dichlorobenzaldehyde, 3,4-dichlorobenzaldehyde, 3,5-dichlorobenzaldehyde, 2,3-difluorobenzaldehyde, 2,4-difluorobenzaldehyde, 2,5-difluorobenzaldehyde, 2,6-difluorobenzaldehyde, 3,4-difluorobenzaldehyde, 3,5-difluorobenzaldehyde, 2,3,4-trifluorobenzaldehyde, 2-(trifluoromethyl)benzaldehyde, 3-(trifluoromethyl) benzaldehyde, 4-(trifluoromethyl) benzaldehyde, 3-(trifluoromethoxy)benzaldehyde, 5-(trifluoromethoxy)salicyl-aldehyde, 3,5-dichlorosalicylaldehyde, 2-amino-benzaldehyde, 2-nitrobenzaldehyde, 3-nitrobenzaldehyde, 4-nitrobenzaldehyde, 3-cyanobenzaldehyde, 4-cyano-benzaldehyde, 4-dimethylamino-1-naphthaldehyde, 4-(dimethylamino)benzaldehyde, 4-(diethylamino)benzaldehyde and 3,4,5-trimethoxybenzaldehyde; 1-naphthaldehyde and 2-naphthaldehyde), heterocyclic (e.g. pyrrole-2-carboxaldehyde, 2-thiophene-carboxaldehyde, 3-thiophene-carboxaldehyde, pyrrolidine-carboxaldehyde and piperonal) or mixed (e.g. phenylacetaldehyde).

Such reaction may be performed in any suitable solvent system for both reagents, such as benzene or toluene. Reaction may usually be effected at the solvent boiling temperature (e.g. between about 80° C. and about 110° C.). Reaction is preferably carried out by using an at least stoechiometric amount, more preferably a molar ratio in the range from about 1.1 to about 3.0, of the aldehyde with respect to the isoquinolinedione derivative.

The present invention further provides the use of a substituted naphthalimide (isoquinolinedione) derivative represented by the general formula (I) or the general formula (II), or a pharmaceutically acceptable salt or a solvate thereof, as a biologically-active ingredient, i.e. an active principle, especially as a medicine or a diagnostic agent or for the manufacture of a medicament or a diagnostic kit. In particular the said medicament may be for the prevention or treatment of a pathologic condition selected from the group consisting of cell proliferative disorders.

The compounds according to this invention are highly active against several types of cancers. Therefore, due to their favorable pharmacological properties, the compounds according to this invention are particularly suitable for use as medicaments or in the preparation of medicaments and combined preparations for the treatment of patients suffering from diseases associated with cell proliferation, more especially for treating cancer.

The term “cell proliferative disorder” as used herein refers to, but is not limited to, any type of cancer or other pathologic condition involving cell proliferation such as leukemia, lung cancer, colorectal cancer, central nervous system (CNS) cancer, melanoma, ovarian cancer, kidney cancer, prostate cancer, breast cancer, glioma, bladder cancer, bone cancer, sarcoma, head and neck cancer, liver cancer, testicular cancer, pancreatic cancer, stomach cancer, oesophaegal cancer, bone marrow cancer, duodenum cancer, eye cancer (retinoblastoma) and lymphoma.

Any of the uses mentioned above may also be restricted to a non-medical use (e.g. in a cosmetic composition), a non-therapeutic use, a non-diagnostic use, a non-human use (e.g. in a veterinary composition), or exclusively an in-vitro use, or a use with cells remote from an animal.

The invention further relates to a pharmaceutical composition comprising:
(a) one or more substituted naphthalimide (isoquinolinedione) derivative represented by the general formula (I) or the general formula (II), and/or a pharmaceutically acceptable salt thereof and/or a solvate thereof, and
(b) one or more pharmaceutically acceptable carriers.

In another embodiment, this invention provides combined preparations, preferably synergistic combinations, of one or more naphthalimide (isoquinolinedione) derivative represented by the general formulae (I) or (II), and/or a pharmaceutically acceptable salt thereof and/or a solvate thereof, with one or more biologically-active drugs being preferably selected from the group consisting of antineoplastic drugs. As is conventional in the art, the evaluation of a synergistic effect in a drug combination may be made by analysing the quantification of the interactions between individual drugs, using the median effect principle described by Chou et al. in Adv. Enzyme Reg. (1984) 22:27. Briefly, this principle states that interactions (synergism, additivity, antagonism) between two drugs can be quantified using the combination index (hereinafter referred as CI) defined by the following equation:

$${CI}_{x} = \frac{{ED}_{x}^{1 c}}{{ED}_{x}^{1 a}} + \frac{{ED}_{x}^{2 c}}{{ED}_{x}^{2 a}}$$

wherein EDₓ is the dose of the first or respectively second drug used alone (1a, 2a), or in combination with the second or respectively first drug (1c, 2c), which is needed to produce a given effect. The said first and second drug have synergistic or additive or antagonistic effects depending upon CI<1, CI=1, or CI>1, respectively. As will be explained in more detail herein-below, this principle may be applied to a number of desirable effects such as, but not limited to, an activity against cell proliferation.

The invention further relates to a composition or combined preparation having synergistic effects against cell proliferation and containing:
(a) one or more antineoplastic drugs, and
(b) at least one naphthalimide (isoquinolinedione) derivative represented by the general formula (I) or the general formula (II), and/or a pharmaceutically acceptable salt thereof and/or a solvate thereof, and
(c) optionally one or more pharmaceutical excipients or pharmaceutically acceptable carriers,
   for simultaneous, separate or sequential use in the treatment or prevention of cell proliferative disorders.

Suitable antineoplastic drugs for inclusion into the synergistic antiproliferative pharmaceutical compositions or combined preparations of this invention are preferably selected from the group consisting of alkaloids, alkylating agents (including but not limited to alkyl sulfonates, aziridines, ethylenimines, methylmelamines, nitrogen mustards and nitrosoureas), antibiotics, antimetabolites (including but not limited to folic acid analogs, purine analogs and pyrimidine analogs), enzymes, interferon and platinum complexes. More specific examples include acivicin; aclarubicin; acodazole; acronine; adozelesin; aldesleukin; altretamine; ambomycin; ametantrone; aminoglutethimide; amsacrine; anastrozole; anthramycin; asparaginase; asperlin; azacitidine; azetepa; azotomycin; batimastat; benzodepa; bicalutamide; bisantrene; bisnafide; bizelesin; bleomycin; brequinar; bropirimine; busulfan; cactinomycin; calusterone; caracemide; carbetimer; carboplatin; carmustine; carubicin; carzelesin; cedefingol; chlorambucil; cirolemycin; cisplatin; cladribine; crisnatol; cyclophosphamide; cytarabine; dacarbazine; dactinomycin; daunorubicin; decitabine; dexormaplatin; dezaguanine; diaziquone; docetaxel; doxorubicin; droloxifene; dromostanolone; duazomycin; edatrexate; eflomithine; elsamitrucin; enloplatin; enpromate; epipropidine; epirubicin; erbulozole; esorubicin; estramustine; etanidazole; ethiodized oil I 131; etoposide; etoprine; fadrozole; fazarabine; fenretinide; floxuridine; fludarabine; fluorouracil; flurocitabine; fosquidone; fostriecin; gemcitabine; Gold 198; hydroxyurea; idarubicin; ifosfamide; ilmofosine; interferon α-2a; interferon α-2b; interferon α-n1; interferon α-n3; interferon β-1a; interferon γ-1b; iproplatin; irinotecan; lanreotide; letrozole; leuprolide; liarozole; lometrexol; lomustine; losoxantrone; masoprocol; maytansine; mechlorethamine; megestrol; melengestrol; melphalan; menogaril; mercaptopurine; methotrexate; metoprine; meturedepa; mitindomide; mitocarcin; mitocromin; mitogillin; mitomalcin; mitomycin; mitosper; mitotane; mitoxantrone; mycophenolic acid; nocodazole; nogala-mycin; ormaplatin; oxisuran; paclitaxel; pegaspargase; peliomycin; pentamustine; peplomycin; perfosfamide; pipobroman; piposulfan; piroxantrone; plicamycin; plomestane; porfimer; porfiromycin; prednimustine; procarbazine; puromycin; pyrazofurin; riboprine; rogletimide; safingol; semustine; simtrazene; sparfosate; sparsomycin; spirogermanium; spiromustine; spiroplatin; streptonigrin; streptozocin; strontium 89 chloride; sulofenur; talisomycin; taxane; taxoid; tecogalan; tegafur; teloxantrone; temoporfin; teniposide; teroxirone; testolactone; thiamiprine; thioguanine; thiotepa; tiazofurin; tirapazamine; topotecan; toremifene; trestolone; triciribine; trimetrexate; triptorelin; tubulozole; uracil mustard; uredepa; vapreotide; verteporfin; vinblastine; vincristine; vindesine; vinepidine; vinglycinate; vinleurosine; vinorelbine; vinrosidine; vinzolidine; vorozole; zeniplatin; zinostatin; zorubicin; and their pharmaceutically acceptable salts.

Other suitable anti-neoplastic compounds include 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; anti-androgens such as, but not limited to, benorterone, cioteronel, cyproterone, delmadinone, oxendolone, topterone, zanoterone; anti-estrogens such as, but not limited to, clometherone; delmadinone; nafoxidine; nitromifene; raloxifene; tamoxifen; toremifene; trioxifene and their pharmaceutically acceptable salts; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin; azasetron; azatoxin; azatyrosine; baccatin III derivatives; balanol; batimastat; BCR/ABL antagonists; benzochlorins; benzoylstaurosporine; β-lactam derivatives; β-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors; castanospermine; cecropin B; cetrorelix; chlorins; chloroquinoxaline sulfonamide; cicaprost; cis-porphyrin; clomifene and analogues thereof; clotrimazole; collismycin A and B; combretastatin and analogues thereof; conagenin; crambescidin 816; cryptophycin and derivatives thereof; curacin A; cyclopentanthraquinones; cycloplatam; cypemycin; cytarabine; cytolytic factor; cytostatin; dacliximab; dehydrodidemnin B; deslorelin; dexifosfamide; dexrazoxane; dexverapamil; didemnin B; didox; diethylnorspermine; dihydro-5-azacytidine; dihydrotaxol; dioxamycin; diphenyl spiromustine; docosanol; dolasetron; doxifluridine; droloxifene; dronabinol; duocarmycin SA; ebselen; ecomustine; edelfosine; edrecolomab; elemene; emitefur; epristeride; estrogen agonists and antagonists; exemestane; filgrastim; finasteride; flavopiridol; flezelastine; fluasterone; fluorodaunorunicin; forfenimex; formestane; fotemustine; gadolinium texaphyrin; gallium nitrate; galocitabine; ganirelix; gelatinase inhibitors; glutathione inhibitors; hepsulfam; heregulin; hexamethylene bisacetamide; hypericin; ibandronic acid; idoxifene; idramantone; ilomastat; imidazoacridones; imiquimod; immunostimulant peptides; insulin-like growth factor-1 receptor inhibitor; interferon agonists; iobenguane; iododoxorubicin; ipomeanol; irinotecan; iroplact; irsogladine; isobengazole; isohomohalicondrin B; itasetron; jasplakinolide; kahalalide F; lamellarin-N; leinamycin; lenograstim; lentinan; leptolstatin; leukemia inhibiting factor; leuprorelin; levamisole; liarozole; lissoclinamide; lobaplatin; lombricine; lonidamine; lovastatin; loxoribine; lurtotecan; lutetium texaphyrin; lysofylline; mannostatin A; marimastat; masoprocol; maspin; matrilysin inhibitors; matrix metalloproteinase inhibitors; merbarone; meterelin; methioninase; metoclopramide; MIF inhibitors; mifepristone; miltefosine; mirimostim; mitoguazone; mitolactol; mitonafide; mitotoxin fibroblast growth factor-saporin; mofarotene; molgramostim; human chorionic gonadotrophin monoclonal antibody; mopidamol; mycaperoxide B; myriaporone; N-acetyldinaline; N-substituted benzamides; nafarelin; nagrestip; naloxone; pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; octreotide; okicenone; onapristone; ondansetron; ondansetron; oracin; osaterone; oxaliplatin; oxaunomycin; palauamine; palmitoylrhizoxin; pamidronic acid; panaxytriol; panomifene; parabactin; pazelliptine; peldesine; pentosan; pentostatin; pentrozole; perflubron; perillyl alcohol; phenazinomycin; phenylacetate; phosphatase inhibitors; picibanil; pilocarpine; pirarubicin; piritrexim; placetin A and B; plasminogen activator inhibitor; propyl bis-acridone; prostaglandin J2; proteasome inhibitors; protein kinase C inhibitors; protein tyrosine phosphatase inhibitors; purine nucleoside phosphorylase inhibitors; purpurins; pyrazoloacridine; raltitrexed; ramosetron; ras farnesyl protein transferase inhibitors; ras inhibitors; ras-GAP inhibitors; retelliptine; rhenium 186 etidronate; rhizoxin; retinamide; rohitukine; romurtide; roquinimex; rubiginone B1; ruboxyl; saintopin; sarcophytol A; sargramostim; sizofiran; sobuzoxane; sodium borocaptate; sodium phenylacetate; solverol; somatomedin binding protein; sonermin; sparfosic acid; spicamycin D; splenopentin; spongistatin 1; squalamine; stem-cell division inhibitors; stipiamide; stromelysin inhibitors; sulfinosine; suradista; suramin; swainsonine; tallimustine; tamoxifen; tauromustine; tazarotene; tecogalan; tellurapyrylium; telomerase inhibitors; temozolomide; tetrachlorodecaoxide; tetrazomine; thaliblastine; thiocoraline; thrombopoietin; thymalfasin; thymopoietin receptor agonist; thymotrinan; thyroid stimulating hormone; tin ethyl etiopurpurin; titanocene; topsentin; tretinoin; triacetyluridine; tropisetron; turosteride; tyrosine kinase inhibitors; tyrphostins; ubenimex; urogenital sinus-derived growth inhibitory factor; urokinase receptor antagonists; variolin B; velaresol; veramine; verdins; verteporfin; vinxaltine; vitaxin; zanoterone; zilascorb; and their pharmaceutically acceptable salts.

Synergistic activity of the pharmaceutical compositions or combined preparations of this invention against cell proliferation may be readily determined by means of one or more tests such as, but not limited to, the measurement of the radioactivity resulting from the incorporation of ³H-thymidine in culture of tumour cell lines. For instance, different tumour cell lines are selected in order to evaluate the anti-tumour effects of the test compounds, such as but not limited to:
RPM11788: human Peripheral Blood Leucocytes (PBL) Caucasian tumor line,
Jurkat: human acute T cell leukemia,
EL4: C57BI/6 mouse lymphoma, or
THP-1: human monocyte tumour line.
   Depending on the selected tumour cell line, different culture media may be used, such as for example:
for RPMI1788 and THP-1: RPMI-1640+10% FCS+1% NEM+1% sodium pyruvate +5×10⁻⁵ mercapto-ethanol+antibiotics (G-418 0.45 μg/ml).
for Jurkat and EL4: RPMI-1640+10% FCS+antibiotics (G-418 0.45 μg/ml).

In a specific embodiment of the synergy determination test, the tumour cell lines are harvested and a suspension of 0.27×10⁶ cells/ml in complete medium is prepared. The suspensions (150 μl) are added to a microtiter plate in triplicate. Either complete medium (controls) or the test compounds at the test concentrations (50 μl) are added to the cell suspension in the microtiter plate. The cells are incubated at 37° C. under 5% CO₂ for about 16 hours. ³H-thymidine is added, and the cells incubated for another 8 hours. The cells are harvested and radioactivity is measured in counts per minute (CPM) in a β-counter. The ³H-thymidine cell content, and thus the measured radioactivity, is proportional to the proliferation of the cell lines. The synergistic effect is evaluated by the median effect analysis method as disclosed herein-before.

The pharmaceutical composition or combined preparation with synergistic activity against cell proliferation according to this invention may contain the naphthalimide (isoquinolinedione) derivative of general formula (I) or (II), and/or a pharmaceutically acceptable salt thereof and/or a solvate thereof, over a broad content range depending on the contemplated use and the expected effect of the preparation. Generally, the naphthalimide (isoquinolinedione) derivative content of the combined preparation is within the range of 0.1 to 99.9% by weight, preferably from 1 to 99% by weight, more preferably from 5 to 95% by weight.

The pharmaceutical compositions and combined preparations according to this invention may be administered orally or in any other suitable fashion. Oral administration is preferred and the preparation may have the form of a tablet, aqueous dispersion, dispersable powder or granule, emulsion, hard or soft capsule, syrup, elixir or gel. The dosing forms may be prepared using any method known in the art for manufacturing these pharmaceutical compositions and may comprise as additives sweeteners, flavoring agents, coloring agents, preservatives and the like. Carrier materials and excipients are detailed hereinbelow and may include, inter alia, calcium carbonate, sodium carbonate, lactose, calcium phosphate or sodium phosphate; granulating and disintegrating agents, binding agents and the like. The pharmaceutical composition or combined preparation of this invention may be included in a gelatin capsule mixed with any inert solid diluent or carrier material, or has the form of a soft gelatin capsule, in which the ingredient is mixed with a water or oil medium. Aqueous dispersions may comprise the biologically active composition or combined preparation in combination with a suspending agent, dispersing agent or wetting agent. Oil dispersions may comprise suspending agents such as a vegetable oil. Rectal administration is also applicable, for instance in the form of suppositories or gels. Injection (e.g. intramuscularly or intraperitoneally) is also applicable as a mode of administration, for instance in the form of injectable solutions or dispersions, depending upon the disorder to be treated and the condition of the patient.

The term “pharmaceutically acceptable carrier or excipient” as used herein in relation to pharmaceutical compositions and combined preparations means any material or substance with which the active principle, i.e. the substituted naphthalimide and optionally the antineoplastic drug, may be formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, pellets or powders.

Suitable pharmaceutical carriers for use in the said pharmaceutical compositions and their formulation are well known to those skilled in the art. There is no particular restriction to their selection within the present invention although, due to the usually low or very low water-solubility of the pteridine derivatives of this invention, special attention will be paid to the selection of suitable carrier combinations that can assist in properly formulating them in view of the expected time release profile. Suitable pharmaceutical carriers include additives such as wetting agents, dispersing agents, stickers, adhesives, emulsifying or surface-active agents, thickening agents, complexing agents, gelling agents, solvents, coatings, antibacterial and antifungal agents (for example phenol, sorbic acid, chlorobutanol), -isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, dissolving, spray-drying, coating and/or grinding the active ingredients, in a one-step or a multi-steps procedure, with the selected carrier material and, where appropriate, the other additives such as surface-active agents. The pharmaceutical compositions of the present invention may also be prepared by micronisation, for instance in view to obtain them in the form of microspheres usually having a diameter of about 1 to 10 μm, namely for the manufacture of microcapsules for controlled or sustained release of the biologically active ingredient(s).

Suitable surface-active agents to be used in the pharmaceutical compositions of the present invention are non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecylbenzene sulphonic acid or dibutyl-naphtalenesulphonic acid or a naphtalenesulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanyl-phosphatidylcholine, dipalmitoylphoshatidylcholine and their mixtures.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/ polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, preferably halides, having 4 hydrocarbon radicals optionally substituted with halo, phenyl, substituted phenyl or hydroxy; for instance quaternary ammonium salts containing as N-substituent at least one C₈-C₂₂ alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbuch”, 2^{nd} ed. (Hanser Verlag, Vienna, 1981) and “Encyclopaedia of Surfactants (Chemical Publishing Co., New York, 1981).

Structure-forming, thickening or gel-forming agents may be included into the pharmaceutical compositions and combined preparations of the invention. Suitable such agents are in particular highly dispersed silicic acid, such as the product commercially available under the trade name Aerosil; bentonites; tetraalkyl ammonium salts of montmorillonites (e.g., products commercially available under the trade name Bentone), wherein each of the alkyl groups may contain from 1 to 20 carbon atoms; cetostearyl alcohol and modified castor oil products (e.g. the product commercially available under the trade name Antisettle).

Gelling agents which may be included into the pharmaceutical compositions and combined preparations of the present invention include, but are not limited to, cellulose derivatives such as carboxymethylcellulose, cellulose acetate and the like; natural gums such as arabic gum, xanthum gum, tragacanth gum, guar gum and the like; gelatin; silicon dioxide; synthetic polymers such as carbomers, and mixtures thereof. Gelatin and modified celluloses represent a preferred class of gelling agents.

Other optional excipients which may be included in the pharmaceutical compositions and combined preparations of the present invention include additives such as magnesium oxide; azo dyes; organic and inorganic pigments such as titanium dioxide; UV-absorbers; stabilisers; odor masking agents; viscosity enhancers; antioxidants such as, for example, ascorbyl palmitate, sodium bisulfite, sodium metabisulfite and the like, and mixtures thereof; preservatives such as, for example, potassium sorbate, sodium benzoate, sorbic acid, propyl gallate, benzylalcohol, methyl paraben, propyl paraben and the like; sequestering agents such as ethylene-diamine tetraacetic acid; flavoring agents such as natural vanillin; buffers such as citric acid and acetic acid; extenders or bulking agents such as silicates, diatomaceous earth, magnesium oxide or aluminum oxide; densification agents such as magnesium salts; and mixtures thereof.

Additional ingredients may be included in order to control the duration of action of the biologically-active ingredient in the compositions and combined preparations of the invention. Control release compositions may thus be achieved by selecting appropriate polymer carriers such as for example polyesters, polyamino-acids, polyvinyl-pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethyl-cellulose, polymethyl methacrylate and the other above-described polymers. Such methods include colloid drug delivery systems like liposomes, microspheres, microemulsions, nanoparticles, nanocapsules and so on. Depending on the route of administration, the pharmaceutical composition or combined preparation of the invention may also require protective coatings.

Pharmaceutical forms suitable for injectable use include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol, complexing agents such as cyclodextrins and the like, and mixtures thereof.

Since, in the case of combined preparations including the substituted naphthalimide (isoquinolinedione) derivative of general formula (I) or (II), and/or a pharmaceutically acceptable salt thereof and/or a solvate thereof, and an antineoplastic drug, both ingredients do not necessarily bring out their synergistic therapeutic effect directly at the same time in the patient to be treated, the said combined preparation may be in the form of a medical kit or package containing the two ingredients in separate but adjacent form. In the latter context, each ingredient may therefore be formulated in a way suitable for an administration route different from that of the other ingredient, e.g. one of them may be in the form of an oral or parenteral formulation whereas the other is in the form of an ampoule for intravenous injection or an aerosol.

The present invention further relates to a method for preventing or treating a cell proliferative disorder in a patient, preferably a mammal, more preferably a human being. The method of this invention consists of administering to the patient in need thereof an effective amount of a substituted naphthalimide (isoquinolinedione) derivative having the general formula (I) or the general formula (II), and/or a pharmaceutically acceptable salt thereof and/or a solvate thereof, optionally together with an effective amount of an antineoplastic drug, or a pharmaceutical composition comprising the same, such as disclosed above in extensive details. The effective amount of the substituted naphthalimide (isoquinolinedione) derivative is usually in the range of 0.01 mg to 20 mg, preferably 0.1 mg to 5 mg, per day per kg bodyweight for humans. Depending upon the pathologic condition to be treated and the patient's condition, the said effective amount may be divided into several sub-units per day or may be administered at more than one day intervals. The patient to be treated may be any warm-blooded animal, preferably a human being, suffering from said pathologic condition.

The following examples are intended to illustrate several embodiments of the present invention, including the preparation and biological evaluation of the substituted naphthalimides, without limiting its scope in any way.

### EXAMPLE 1

### Preparation of 2-chloro-N-[({2-[2-(dimethylamino)ethyl]-1,3-dioxo-2,3-dihydro-1H-benzo[de]isoquinolin-5-yl}amino)carbonyl]acetamide

100 mg of amonafide were dissolved in 2 mL of acetonitrile under nitrogen atmosphere, then 95 mg of 2-chloroacetyl isocyanate (2 equivalents) in 2.5 mL of acetonitrile were carefully added. Reaction was maintained at room temperature for 4 hours. Acetonitrile was then evaporated under reduced pressure and the residue was submitted to a flash chromatography (SiO₂, eluent : CH₂Cl₂/MeOH 95:5), thus resulting in 25.5 mg (yield: 18%) of the desired product: 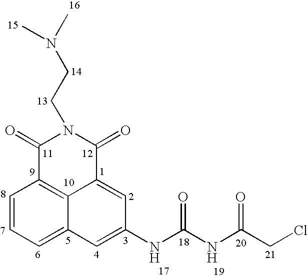
which was characterised by proton nuclear magnetic resonance (hereinafter referred as ¹H NMR) performed at 300 MHz in DMSO, as follows: 11.15 (H-17, bs); 10.30 (H-19, s); 8.57 (H-2, d, J=2.1); 8.53 (H-4, d, J=2.1); 8.34 (H-8, s); 8.32 (H-6, s); 7.79 (H-7, t, J=7.8); 4.17 (H-13, t, J=6.8), 3.76 (H-21, s); 2.61 (H-14, t, J=6.6) and 2.29 (H-15 and H-16, s).

### EXAMPLE 2

### Preparation of 2,2,2-trichloro-N-[({2-[2-(dimethylamino)ethyl]-1,3-dioxo-2,3-dihydro-1H-benzo[de]isoquinolin-5-yl}amino)carbonyl]acetamide

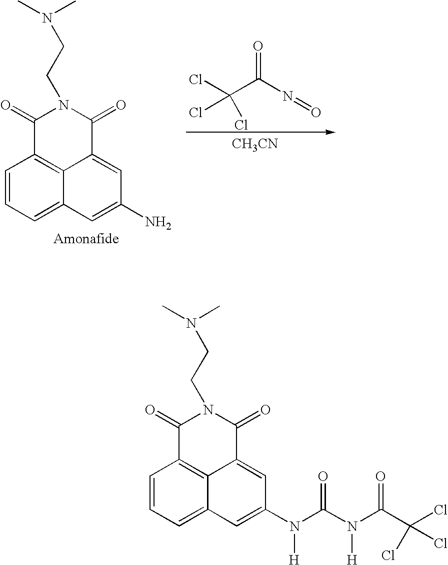

700 mg of amonafide were dissolved in 14 mL of acetonitrile under nitrogen atmosphere. 932 mg of trichloroacetyl isocyanate (2 equivalents) in 14 mL of acetonitrile were carefully added. Reaction was maintained at room temperature for 4.5 hours. Acetonitrile was then evaporated under reduced pressure and the residue was submitted to a flash chromatography (SiO₂, eluent : CH₂Cl₂/MeOH 97:3), thus resulting in 540.5 mg (yield: 46%) of the desired product which was characterised by:
hu 1H NMR (300 MHz, DMSO) as follows:11.18 (H-17and H-19, bs); 8.76 (H-2, bs); 8.75 (H-4, bs); 8.43 (H-8, d, J=6.6); 8.41 (H-6, d, J=6.0); 7.85 (H-7, t, J=7.5); 4.20 (H-13, t, J=6.6), 2.69 (H-14, t, J=6.3) and 2.35 (H-15 and H-16, s) (same atom numbering as in example 1), and
carbon nuclear magnetic resonance (hereinafter referred as ¹³C NMR) performed at 300 MHz in DMSO, as follows: 163.2 and 162.9 (C-11 and C-12); 160.1 (C-18); 149.8 (C-20); 136.0; 134.0; 131.6; 129.9; 127.7; 125.0; 124.7; 123.9; 122.7; 121.8; 79.1 (C-21); 56.0 (C-14); 44.8 (C-15 and C-16) and 37.1 (C-13).

### EXAMPLE 3

### Preparation of N-[({2-[2-(dimethylamino)ethyl]-1.3-dioxo-2,3-dihydro-1H-benzo[de]isoquinolin-5-yl}amino)carbonyl]benzamide

100 mg of amonafide were dissolved in 2 mL of acetonitrile under nitrogen atmosphere. 105 mg of benzoyl isocyanate (2 equivalents) in 2.5 mL of acetonitrile were carefully added. Reaction was maintained at room temperature for 4 hours. The solvent was then evaporated under reduced pressure and the residue was submitted to a flash chromatography (SiO₂, eluent: CH₂Cl₂/MeOH 95:5), thus resulting in 140.2 mg (yield: 92%) of the desired product: 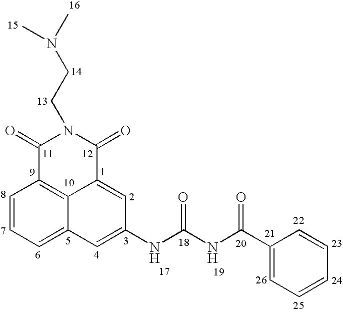
which was characterised by ¹H NMR (300 MHz, DMSO) as follows 11.25 (H-17 and H-19, bs); 8.72 (H-2, d, J=2.1); 8.60 (H-4, d, J=2.1); 8.39 (H-8, d, J=2.7); 8.36 (H-6, d, J=1.5); 8.06 (H-22 and H-26, d, J=7.2); 7.83 (H-7, t, J=7.6); 7.69 (H-24, t, J=7.5); 7.57 (H-23 and H-25, t, J=8.1); 4.17 (H-13, t, J=6.9), 2.55 (H-14, t, J=6.9) and 2.24 (H-15 and H-16, s).

### EXAMPLE 4

### Preparation of ethyl({2-[2-(dimethylamino)ethyl]-1.3-dioxo-2,3-dihydro-1H-benzo[de]isoquinolin-5-yl}amino)carbonylcarbamate

100 mg of amonafide were dissolved in 2 mL of acetonitrile under nitrogen atmosphere. 82 mg of ethoxycarbonyl isocyanate (2 equivalents) in 2.5 mL of acetonitrile were carefully added. Reaction was maintained at room temperature for 4 hours. Acetonitrile was then evaporated under reduced pressure and the residue was submitted to a flash chromatography (SiO₂, eluent: CH2Cl2/MeOH 95:5), thus resulting in 46.6 mg (yield: 33%) of the desired product 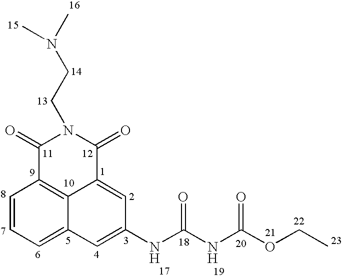
which was characterised by ¹H NMR (300 MHz, CDCl₃) as follows: 8.70 (H-2, d, J=2.1); 8.42 (H-8, d, J=7.2); 8.28 (H-4, d, J=2.1); 8.13 (H-6, d, J=7.5); 7.68 (H-7, t, J=7.6); 4.35 (H-13, t, J=6.6); 4.11 (H-22, q, J=7.5); 2.79 (H-14, t, J=6.8); 2.41 (H-15 and H-16, s) and 1.30 (H-23, t, J=7.2).

### EXAMPLE 5

### Preparation of N-[2-[2-(dimethylamino)ethyl]-1,3-dioxo-2,3-dihydro-1H-benzo[de]isoquinolin-5-yl]-N′-(2-chloroethyl)urea

400 mg of amonafide were dissolved in 8 mL of acetonitrile under nitrogen atmosphere. 299 mg of 2-chloroethylisocyanate (2 equivalents) in 8 mL of acetonitrile were carefully added. Reaction was maintained at room temperature for 72 hours. Acetonitrile was then evaporated under reduced pressure and the residue was submitted to a flash chromatography (SiO₂, eluent : CH₂Cl₂/MeOH 95:5), thus resulting in 465.9 mg (yield: 85%) of the desired product 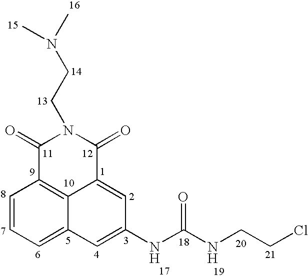
which was characterised by:
¹H NMR (300 MHz, DMSO) as follows: 9.39 (H-17, s); 8.53 (H-2, bs); 8.49 (H-4, bs); 8.30 (H-8, bs); 8.28 (H-6, bs); 7.77 (H-7, t, J=7.8); 6.65 (H-19, t, J=5.4); 4.16 (H-13, t, J=6.6), 3.72 (H-21, t, J=6.2); 3.49 (H-20, quadr, J=6.0 and 5.7); 2.55 (H-14, t, J=6.6) and 2.24 (H-15 and H-16, s), and
¹³C NMR (300 MHz, DMSO) as follows: 163.3 and 163.1 (C-11 and C-12); 154.9 (C-18); 139.2 (C-3); 133.1; 132.2; 128.0; 127.2; 123.1; 122.9; 122.3; 121.6; 118.5; 56.3 (C-14); 54.7 (C-20); 45.2 (C-15 and C-16); 44.0 (C-21) and 37.4 (C-13).

### EXAMPLE 6

### Preparation of N-[2-[2-(dimethylamino)ethyl]-1,3-dioxo-2,3-dihydro-1H-benzo[de]isoquinolin-5-yl]-N′-(4-chlorophenyl)urea

100 mg of amonafide were dissolved in 2 mL of acetonitrile under nitrogen atmosphere. 109 mg of 4-chlorophenyl isocyanate (2 equivalents) in 2.5 mL of acetonitrile were carefully added. Reaction was maintained at room temperature for 4 hours. Acetonitrile was then evaporated under reduced pressure and the residue was submitted to a flash chromatography (SiO₂, eluent : CH2Cl2/MeOH 95:5), thus resulting in 102.2 mg (yield: 66%) of the desired product 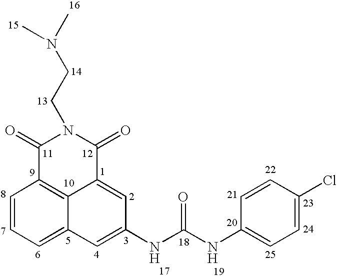
which was characterised by:
¹H NMR (300 MHz, DMSO) as follows: 9.41 (H-19, s); 9.04 (H-17, s); 8.56 (H-2, d, J=1.8); 8.52 (H-4, d, J=2.1); 8.34 (H-8, d, J=3.0); 8.32 (H-6, d, J=1.8); 7.79 (H-7, t, J=7.8); 7.55 (H-21 and H-25, d, J=9.0); 7.36 (H-22 and H-24, d, J=9.0); 4.16 (H-13, t, J=7.0), 2.50-2.55 (H-14, m) and 2.22 (H-15 and H-16, s), and
¹³C NMR (300 MHz, DMSO) as follows: 163.2 and 163.0 (C-11 and C-12); 152.3 (C-18); 138.4 and 138.2 (C-3 and C-20); 133.2; 132.1; 128.5; 128.3; 127.3; 125.6; 123.4; 123.2; 122.4; 121.6; 119.9; 119.3; 56.3 (C-14); 45.2 (C-15 and C-16) and 37.5 (C-13).

### EXAMPLE 7

### Preparation of N-[2-[2-(dimethylamino)ethyl]-1,3-dioxo-2,3-dihydro-1H-benzo[de]isoquinolin-5-yl]-N′-(4-cyanophenyl)urea

100 mg of amonafide were dissolved in 2 mL of acetonitrile under nitrogen atmosphere. 105 mg of 4-cyanophenyl isocyanate (2 equivalents) in 2.5 mL of acetonitrile were carefully added. Reaction was maintained at room temperature for 4 hours. Acetonitrile was then evaporated under reduced pressure and the residue was submitted to a flash chromatography (SiO₂, eluent: CH2Cl2/MeOH 95:5), thus resulting in 131.8 mg (yield: 87%) of the desired product: 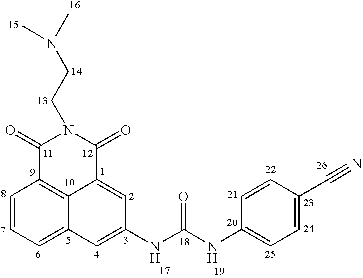
which was characterised by ¹H NMR (300 MHz, DMSO) as follows: 9.56 (H-19, s); 9.44 (H-17, s); 8.58 (H-2, d, J=2.4); 8.53 (H-4, d, J=2.4); 8.36 (H-8, d, J=4.2); 8.34 (H-6, d, J=3.3); 7.81 (H-7, t, J=7.8); 7.65-7.80 (H-21, H-22, H-24 and H-25, m); 4.17 (H-13, t, J=6.9), 2.54 (H-14, t, J=6.6) and 2.23 (H-15 and H-16, s).

### EXAMPLE 8

### Preparation of ethyl 4-N-[({2-[2-(dimethylamino)ethyl]-1,3-dioxo-2,3-dihydro-1H-benzo[de]isoquinolin-5-yl)amino]carbonyl}amino)benzoate

800 mg of amonafide were dissolved in 15 mL of acetonitrile under nitrogen atmosphere. 1.08 g of ethyl-4-cyanatobenzoate (2 equivalents) in 15 mL of acetonitrile were carefully added. Reaction was maintained at room temperature for 16 hours. Acetonitrile was then evaporated under reduced pressure and the residue was submitted to a flash chromatography (SiO₂, eluent: CH2Cl2/MeOH 90:10), thus resulting in 969.9 mg (yield: 72% of the desired product 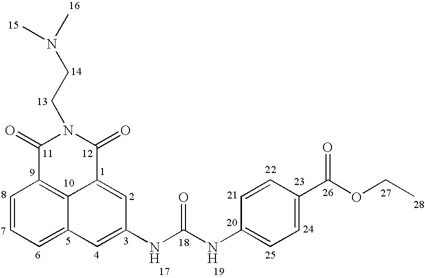
which was characterised by ¹H NMR (300 MHz, DMSO) as follows : 9.63 (H-19, s); 9.45 (H-17, s); 8.58 (H-2, bs); 8.56 (H-4, bs); 8.36 (H-8, d, J=7.5); 8.33 (H-6, d, J=6.6); 7.92 (H-22 and H-24, d, J=8.4); 7.80 (H-7, t, J=7.8); 7.66 (H-21 and H-25, d, J=8.7); 4.29 (H-27, q, J=6.9); 4.17 (H-13, t, J=6.6), 2.54 (H-14, t, J=7.5); 2.23 (H-15 and H-16, s) and 1.32 (H-28, t, J=7.2).

### EXAMPLE 9

### Preparation of N-[({2-[2-(dimethylamino)ethyl]-1,3-dioxo-2,3-dihydro-1H-benzo[de]isoquinolin-5-yl-N′-1,3-benzodioxol-5-yl-urea

100 mg of amonafide were dissolved in 2 mL of acetonitrile under nitrogen atmosphere. 115 mg of 3,4-(methylenedioxy)phenyl isocyanate (2 equivalents) in 2 mL of acetonitrile were carefully added. Reaction was maintained at room temperature for 16 hours. Acetonitrile was then evaporated under reduced pressure and the residue was submitted to a flash chromatography (SiO₂, eluent: CH2Cl2/MeOH 95:5), thus resulting in 152.3 mg (yield: 76%) of the desired product: 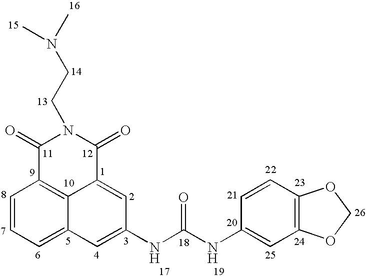
which was characterised by:
¹ H NMR (300 MHz, DMSO) as follows: 9.38 (H-19, s); 8.85 (H-17, s); 8.55 (H-2, d, J=2.4); 8.50 (H-4, d, J=2.4); 8.33 (H-8, d, J=3.0); 8.30 (H-6, d, J=1.8); 7.78 (H-7, t, J=7.6); 7.26 (H-21, d, J=1.8); 6.84-6.86 (H-22 and H-25, bs); 5.99 (H-26, s); 4.15 (H-13, t, J=6.7), 2.53 (H-14, m) and 2.21 (H-15 and H-16, s); and
¹³C NMR (300 MHz, DMSO) as follows: 163.3 and 163.0 (C-11 and C-12); 152.5 (C-18); 147.1; 142.2; 138.7; 133.6; 133.2; 132.2; 128.2; 127.3; 123.3; 123.1; 122.3; 119.1; 111.3; 108.0; 101.1; 100.7; 56.3 (C-14); 45.3 (C-15 and C-16) and 37.5 (C-13).

### EXAMPLE 10

### Preparation of N-[({2-[2-(dimethylamino)ethyl]-1,3-dioxo-2,3-dihydro-1H-benzo[de]isoquinolin-5-yl]-N′-[4-(trifluoromethoxy)phenyl]urea

450 mg of amonafide were dissolved in 9 mL of acetonitrile under nitrogen atmosphere. 645 mg of 4-(trifluoromethoxy)phenyl isocyanate (2 equivalents) in 9 mL of acetonitrile were carefully added. Reaction was maintained at room temperature for 3 hours. Acetonitrile was then evaporated under reduced pressure and the residue was submitted to a flash chromatography (SiO₂, eluent: CH₂Cl₂/MeOH 95:5), thus resulting in 533.6 mg (yield: 69%) of the desired product: 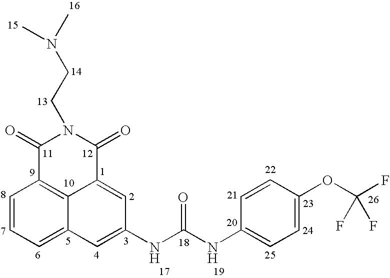
which was characterised by:
¹H NMR (300 MHz, DMSO) as follows: 9.42 (H-19, s); 9.11 (H-17, s); 8.57 (H-2, d, J=2.1); 8.52 (H-4, d, J=1.8); 8.34 (H-8, d, J=3.6); 8.32 (H-6, d, J=2.7); 7.79 (H-7, t, J=7.8); 7.63 (H-21 and H-25, d, J=8.7); 7.32 (H-22 and H-24, d, J=9.0); 4.16 (H-13, t, J=6.6), 2.50-2.55 (H-14, m) and 2.22 (H-15 and H-16, s); and
¹³C NMR (300 MHz, DMSO) as follows: 163.2 and 163.0 (C-11 and C-12); 152.4 (C-18); 142.7; 138.5; 138.4; 133.2; 132.1; 128.3; 127.3; 123.3; 122.4; 121.6; 119.6; 119.4; 56.3 (C-14); 45.2 (C-15 and C-16) and 37.5 (C-13).

### EXAMPLE 11

### Preparation of ethyl 4-N-[({2-[2-(dimethylamino)ethyl]1,3-dioxo-2,3-dihydro-1H-benzo[de]isoquinolin-5-yl]amino}-4-oxobutanoate

100 mg of amonafide were dissolved in 2 mL of acetonitrile under nitrogen atmosphere. 116 mg of ethylsuccinyl chloride (2 equivalents) in 2 mL of acetonitrile were carefully added. Reaction was maintained at room temperature for 1 hour. Acetonitrile was then evaporated under reduced pressure and the residue was submitted to a flash chromatography (SiO₂, eluent : CH2Cl2/MeOH 95:5), thus resulting in 155.1 mg (yield: 100%) of the desired product 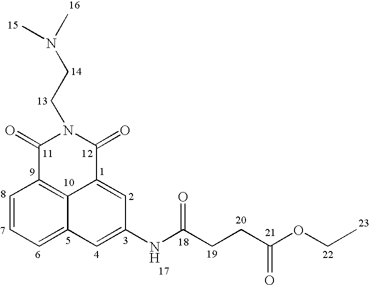
which was characterised by:
¹H NMR (300 MHz, DMSO) as follows: 10.73 (H-17, s); 8.76 (H-2, d, J=1.8); 8.64 (H-4, d, J=2.1); 8.37 (H-8, d, J=2.1); 8.34 (H-6, bs); 7.81 (H-7, t, J=7.8); 4.21 (H-13, t, J=6.6), 4.08 (H-22, q, J=7.2); 2.6-2.8 (H-19, H-20 and H-14, m); 2.38 (H-15 and H-16, s) and 1.20 (H-23, t, J=7.2); and
¹³C NMR (300 MHz, DMSO) as follows: 172.2 (C-18); 170.7 (C-21); 163.3 and 163.3 (C-11 and C-12); 137,9 (C-3); 133.6; 132.0; 128.7; 127.4; 123.8; 123.7; 122.4; 121.7; 120.5; 59.8 (C-22); 55.3 (C-14); 43.8 (C-15 and C-16); 36.2 (C-13); 30.9 (C-20); 28.5 (C-19) and 14.0 (C-23).

### EXAMPLE 12

### Preparation of 4-chloro-N-{2-[2-(dimethylamino)ethyl]-1,3-dioxo-2,3-dihydro-1H-benzo[de]isoquinolin-5-yl}butanamide

100 mg of amonafide were dissolved in 2 mL of acetonitrile under nitrogen atmosphere. 100 mg of 4-chlorobutyryl chloride (2 equivalents) in 2 mL of acetonitrile were carefully added. Reaction was maintained at room temperature for 16 hours. Acetonitrile was then evaporated under reduced pressure and the residue was submitted to a flash chromatography (SiO₂, eluent : CH2Cl2/MeOH 95:5), thus resulting in 155.1 mg (yield: 64%) of the desired product: 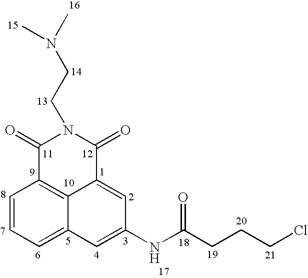
which was characterised by:
¹H NMR (300 MHz, DMSO) as follows: 10.67 (H-17, s); 8.77 (H-2, d, J=2.1); 8.63 (H-4, d, J=2.1); 8.37 (H-8, d, J=3.0); 8.34 (H-6, bs); 7.81 (H-7, t, J=7.8); 4.23 (H-13, m), 3.76 (H-21, t, J=6.3); 2.76 (H-19, m); 2.61 (H-14, t, J=7.4); 2.40 (H-15 and H-16, s) and 2.10 (H-20, t, J=6.9); and
¹³C NMR (300 MHz, DMSO) as follows: 163.5 and 163.5 (C-11 and C-12); 137.9 (C-3); 133.6; 131.9; 128.8; 127.4; 125.2; 123.8; 121.7; 120.7; 55.4 (C-14); 44.8 (C-15 and C-16); 44.0 (C-21); 33.4 (C-13) and 27.8 (C-20).

### EXAMPLE 13

### Preparation of 2-(4-chlorophenyl)-N-{2-[2-(dimethylamino)ethyl]-1,3-dioxo-2,3-dihydro-1H-benzo[de]isoquinolin-5-yl}acetamide

200 mg of amonafide were dissolved in 4 mL of acetonitrile under nitrogen atmosphere. 270 mg of 4-chlorobenzeneacetyl chloride (2 equivalents) in 4 mL acetonitrile were carefully added. Reaction was maintained at room temperature for 16 hours. Acetonitrile was then evaporated under reduced pressure and the residue was submitted to a flash chromatography (SiO₂, eluent: CH₂Cl₂/MeOH 90:10) thus resulting in 311 mg (yield: 100%) of the desired product: 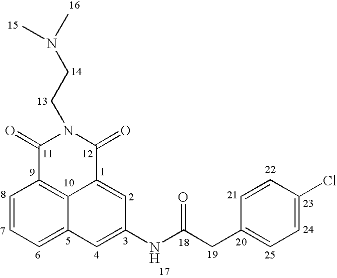
which was characterised by:
¹H NMR (300 MHz, DMSO) as follows :_{—}10.93 (H-17, s); 8.77 (H-2, d, J=2.4); 8.73 (H-4, d, J=1.8); 8.35 (H-8, bs); 8.32 (H-6, d, J=2.7); 7.79 (H-7, t, J=7.8); 7.46 (H-21 and H-25, d, J=8.7); 7.40 (H-22 and H-24, d, J=8.4); 4.35 (H-13, t, J=6.2), 3.84 (H-19, s); 3.29 (H-13, t, J=5.7) and 2.77 (H-15 and H-16, s); and
¹³C NMR (300 MHz, DMSO) as follows: 169.6 (C-18); 163.6 and 163.5 (C-11 and C-12); 137.9 (C-3); 134.7; 133.7; 131.9; 131.3; 131.0; 128.8; 128.1; 127.4; 124.0; 123.8; 122.6; 121.8; 120.8; 54.7 (C-14); 48.5; 42.9 (C-15 and C-16); 42.2 and 35.4 (C-13).

### EXAMPLE 14

### Preparation of ethyl 3-({2-[2-(dimethylamino)ethyl]-1,3-dioxo-2,3-dihydro-1H-benzo[de]isoquinolin-5-yl}amino)-3-oxopropanoate

200 mg of amonafide were dissolved in 4 mL of acetonitrile under nitrogen atmosphere. 235 mg of ethylmalonyl chloride (2 equivalents) in 4 mL of acetonitrile were carefully added. Reaction was maintained at room temperature for 16 hours. Acetonitrile was then evaporated under reduced pressure and the residue was submitted to a flash chromatography (SiO₂, eluent : CH₂Cl₂/MeOH 90:10), thus resulting in 280.1 mg (yield: 100%) of the desired product: 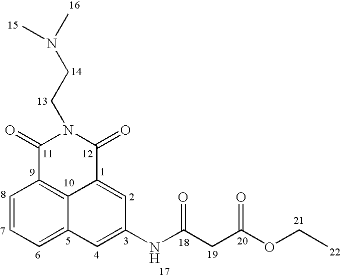
which was characterised by:
¹H NMR (300 MHz, DMSO) as follows: 11.14 (H-17, s); 8.74 (H-2, d, J=2.1); 8.65 (H-4, d, J=1.8); 8.38 (H-8, d, J=5.1); 8.35 (H-6, d, J=4.5); 7.81 (H-7, t, J=8.0); 4.26 (H-13, t, J=6.6), 4.16 (H-21, q, J=6.9); 3.64 (H-19, s); 2.93 (H-14, t, J=6.3); 2.4-2.6 (H-15 and H-16, s) and 1.21 (H-22, m); and
¹³C NMR (300 MHz, DMSO) as follows: 167.4 and 164.9 (C-18 and C-20); 163.5 and 163.2 (C-11 and C-12); 137,5 (C-3); 133.7; 131.9; 129.0; 127.5; 124.1; 123.6; 122.6; 121.8; 120.9; 60.6 (C-21); 60.3 (C-19); 54.8 (C-14); 44.0 (C-15 and C-16); 36.4 (C-13) and 13.9 (C-22).

### EXAMPLE 15

### Preparation of 2-(4-methoxyphenyl)-N-{2-[2-(dimethyl-amino)ethyl]-1,3-dioxo-2,3-dihydro-1H-benzo[de]isoquinolin-5-yl}acetamide

250.0 mg of amonafide were dissolved in 5 mL of acetonitrile under nitrogen atmosphere. 333 mg of 4-methoxyphenylacetyl chloride (2 equivalents) in 5 mL of acetonitrile were carefully added. Reaction was maintained at room temperature for 1 hour. Acetonitrile was then evaporated under reduced pressure and the residue was submitted to a flash chromatography (SiO₂, eluent : CH₂Cl₂/MeOH 90:10), thus resulting in 221.0 mg (yield : 58%) of the desired product: 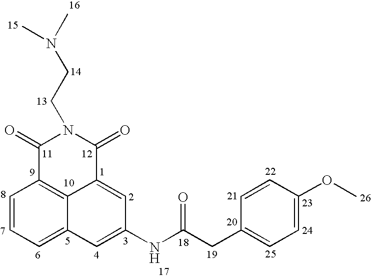
which was characterised by:
¹H NMR (300 MHz, DMSO) as follows 11.34 (H-17, s); 8.80 (H-2, d, J=2.1); 8.76 (H-4, d, J=1.8); 8.35 (H-8, d, J=2.7); 8.32 (H-6, d, J=4.2); 7.79 (H-7, t, J=8.0); 4.30 (H-13, t, J=6.2), 4.19 (H-19, bs); 3.73 (H-16, s); 2.61 (H-15 and H-16, s) and 2.4-2.6 (H-14, m); and
¹³C NMR (75.4 MHz, DMSO) as follows: 170.4 (C-18); 163.6 and 158.0 (C-11 and C-12); 138.2 (C-3); 133.6; 131.9; 130.1; 128.8; 127.7; 137.4; 124.0; 122.6; 121.8; 120.8; 113.7; 54.9 (C-26); 54.8 (C-14); 48.5 (C-15 and C-16) and 43.5 (C-13).

### EXAMPLE 16

### Preparation of 2,2,2-trichloro-N-{2-[2-(dimethylamino)ethyl]-1,3-dioxo-2,3-dihydro-1H-benzo[de]isoquinolin-5-yl}acetamide

250 mg of amonafide were dissolved in 5 mL of acetonitrile under nitrogen atmosphere. 331 mg of trichloroacetyl chloride (2 equivalents) in 5 mL of acetonitrile were carefully added. Reaction was maintained at room temperature for 1 hour. Acetonitrile was then evaporated under reduced pressure and the residue was submitted to a flash chromatography (SiO₂, eluent : CH₂Cl₂/MeOH 90:10), thus resulting in 364.9 mg (yield : 96%) of the desired product: 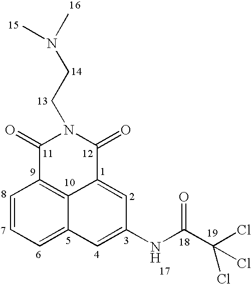
which was characterised by:
¹H NMR (300 MHz, DMSO) as follows: 11.34 (H-17, s); 8.73 (H-2, bs); 8.72 (H-4, bs); 8.41 (H-8, d, J=5.1); 8.38 (H-6, d, J=4.2); 8.35 (H-17, s); 7.83 (H-7, t, J=7.8); 4.16 (H-13, t, J=6.8); 2.50-2.55 (H-14, m) and 2.22 (H-15 and H-16, s); and
¹³C NMR (75.4 MHz, DMSO) as follows: 163.2 and 163.0 (C-1I and C-12); 160.7 (C-18); 133.8 (C-3); 131.7; 129.4; 127.5; 126.0; 124.4; 124.1; 122.4; 121.7; 79.1; 56.3 (C-14); 45.3 (C-15 and C-16) and 37.5 (C-13).

### EXAMPLE 17

### Preparation of 5-{[(1Z)-1,3-benzodioxol-5-ylmethylene]amino}-N-{2-[2-(dimethylamino)ethyl]-1H-benzo[de]isoquinoline-1,3(2H)-dione

A mixture of 300 mg amonafide, 35 mL benzene and 193 mg piperonal (1.2 equivalent) was refluxed for 54 hours. After cooling, benzene was evaporated under reduced pressure and the residue was submitted to a flash chromatography (SiO₂, eluent : CH₂Cl₂/MeOH 90:10), thus resulting in 391 mg (yield: 89%) of the desired product: 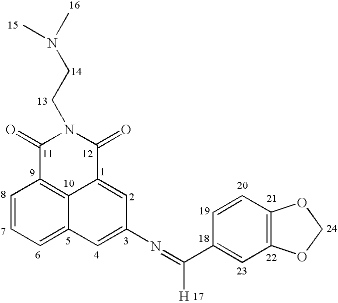
which was characterised by:
¹H NMR (300 MHz, DMSO) as follows: 8.74 (H-17, s); 8.41 (H-2, bs); 8.39 (H-4, bs); 8.33 (H-8, bs); 8.21 (H-6, bs); 7.84 (H-7, t, J=7.5); 7.55 (H-23, bs); 7.51 (H-20, d, J=8.0); 7.09 (H-19, d, J=8.1); 6.15 (H-24, s); 4.16 (H-13, t, J=6.8), 2.50-2.55 (H-14, m) and 2.21 (H-15 and H-16, s); and
¹³C NMR (75.4 MHz, DMSO) as follows: 163.2 and 163.2 (C-11 and C-12); 162.0 (C17); 149.7 (C-21); 148.0 (C-22); 133.9; 132.3; 130.4; 129.6; 127.5; 126.4; 125.0; 123.0; 121.9; 108.4; 106.3; 101.8; 56.3 (C-14); 45.3 (C-15 and C-16) and 37.5 (C-13).

### EXAMPLE 18

### Preparation of 5-{[(1Z)-(4-hydroxy-3-methoxyphenyl)methylene]amino}-2-[2-(dimethylamino)ethyl]l-1H-benzo[de]isoquinoline-1,3(2H)-dione

A mixture of 300 mg amonafide, 10 mL toluene and 195 mg vanillin (152.2 g/mol; 1.2 eq) was refluxed for 16 hours. After cooling, toluene was evaporated under reduced pressure and the residue was submitted to a flash chromatography (SiO₂, eluent: CH₂Cl₂/MeOH 90:10), thus resulting in 408.1 mg (yield: 92%) of the desired product: 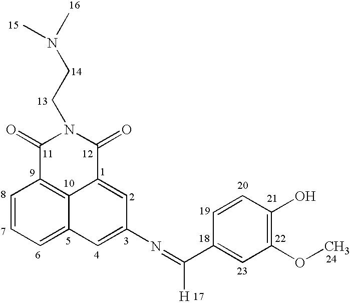
which was characterised by:
¹H NMR (300 MHz, DMSO) as follows: 8.69 (H-2, bs); 8.40 (H-17, s); 8.38 (H-4, bs); 8.32 (H-8, d, J=1.8); 8.19 (H-6, d, J=2.4); 7.83 (H-7, t, J=7.8); 7.62 (H-23, d, J=1.8); 7.44 (H-19, dd, J=1.8 and 8.1); 6.94 (H-20, d, J=8.1); 4.16 (H-13, t, J=6.8); 3.88 (H-24, s); 2.53 (H-14, t, J=6.9) and 2.22 (H-1 5 and H-16, s) ; and
¹³C NMR (75.4 MHz, DMSO): 163.2 and 163.0 (C-11 and C-12); 162.4 (C17); 150.7 (C-22); 150.2 (C-21); 133.8; 132.4; 129.4; 127.5; 127.4; 125.1; 124.6; 124.0; 122.9; 121.9; 115.3; 110.6; 56.3 (C-14); 55.4 (C-24); 45.3 (C-15 and C-16) and 37.5 (C-13).

### EXAMPLE 19

### Preparation of 2-[2-(dimethylamino)ethyl]-5-{[(1Z)-(2,5-dihydroxyphenyl)methylene]amino}-1H-benzo[de]isoquinoline-1,3(2H)-dione

A mixture of 200 mg amonafide, 10 mL toluene and 119 mg 2,5-dihydroxybenzaldehyde (1.2 equivalent) was refluxed for 2 hours. After cooling, toluene was evaporated under reduced pressure and the residue was submitted to a flash chromatography (SiO₂, eluent : CH₂Cl₂/MeOH 90:10), thus resulting in 210.2 mg (yield : 74%) of the desired product: 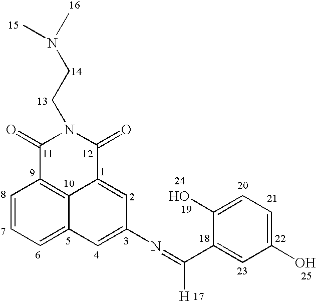
which was characterized by ¹H NMR (300 MHz, DMSO) as follows 11.87 (H-17, s); 9.18 and 9.12 (H-24 and H-25); 8.50 (H-2, bs); 8.46 (H-4, bs); 8.44 (H-8, bs); 8.40 (H-6, bs); 7.89 (H-7, t, J=7.6); 7.16 (H-23, d, J=2.1); 6.94 (H-21, td, J=2.1 and 8.4); 6.90 (H-20, t, J=9.0); 4.18 (H-13, t, J=6.6), 2.55 (H-14, m) and 2.23 (H-15 and H-16, s).

### EXAMPLE 20

### Preparation of 2-[2-(dimethylamino)ethyl]-5-{[(1Z)-(3,4,5-trimethoxyphenyl)methylene]amino}-1H-benzo[de]isoquinoline-1,3(2H)-dione

A mixture of 200 mg amonafide, 10 mL toluene and 168 mg 3,4,5-trimethoxybenzaldehyde (1.2 equivalent) was refluxed for 26 hours. After cooling, toluene was evaporated under reduced pressure and the residue was submitted to a flash chromatography (SiO₂, eluent: CH₂Cl₂/MeOH 95:5), thus resulting in 268.3 mg (yield : 82%) of the desired product: 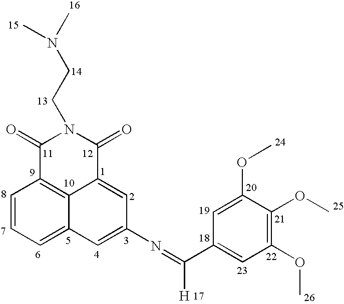
which was characterized by:
¹H NMR (300 MHz, DMSO) as follows: 8.74 (H-17, s); 8.38 (H-2, bs); 8.35 (H-4, bs); 8.30 (H-8, bs); 8.19 (H-6, bs); 7.82 (H-7, t, J=7.8); 7.35 (H-19 and H-23, s); 4.14 (H-13, t, J=6.8), 3.89 (H-24 and H-26, s); 3.77 (H-25, s); 2.52 (H-14, m) and 2.22 (H-15 and H-16, s); and
¹³C NMR (75.4 MHz, DMSO) as follows: 163.0 and 162.9 (C-11 and C-12); 162.4 (C17); 153.1 (C-20 and C-22); 149.4 (C-21); 140.7 (C-3); 133.9 (C-18); 132.2; 131.0; 129.6; 127.5; 125.4; 124.6; 122.9; 121.8; 106.1 (C-19 and C-23); 60.0 (C-25); 56.3 (C-14); 55.8 (C-24 and C-26); 45.3 (C-15 and C-16) and 37.5 (C-13).

### EXAMPLE 21

### Preparation of N-[({2-[2-(dimethylamino)ethyl]-1,3-dioxo-2,3-dihydro-1H-benzo[de]isoquinolin-5-yl]-N′-[4-(trifluoromethoxy)phenyl]thiourea

200 mg of amonafide were dissolved in 4 mL of acetonitrile under nitrogen atmosphere. 320 mg of 4-(trifluoromethoxy)phenyl isothiocyanate (2 equivalents) in 4 mL of acetonitrile were carefully added. Reaction was maintained at room temperature for 6 hours. Then, place the mixture at 50° C. for the week-end. Acetonitrile was then evaporated under reduced pressure and the residue was submitted to a flash chromatography (SiO₂, eluent : CH₂Cl₂/MeOH 95:5), thus resulting in 233.6 mg (yield : 66%) of the desired product: 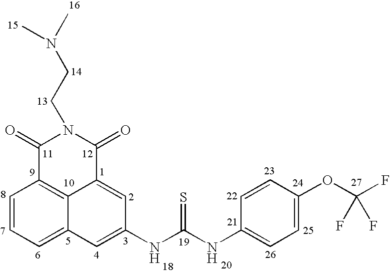
which was characterized by:
¹H NMR (300 MHz, DMSO) as follows: 10.34 (H-18 and H-20, bs); 8.67 (H-2, d, J=2.4); 8.47 (H-4, d, J=1.8); 8.40 (H-8, d, J=6.0); 8.38 (H-6, d, J=7.5); 7.82 (H-7, t, J=7.8); 7.66 (H-23 and H-25, d, J=8.7); 7.37 (H-22 and H-26, d, J=8.1); 4.17 (H-13, t, J=6.8); 2.53 (H-14, t, J=7.2) and 2.23 (H-15 and H-16, s); and
¹³C NMR (75.4 MHz, DMSO) as follows: 180.1 (C-19); 163.2 and 163.0 (C-11 and C-12); 144.7 (C-24); 138.3; 133.6; 131.6; 129.5; 128.3; 127.4; 126.2; 125.2; 124.6; 121.7; 121.8; 121.2; 118.3; 56.3 (C-14); 45.2 (C-15 and C-16) and 37.5 (C-13).

### EXAMPLE 22

### Preparation of N-[({2-[2-(dimethylamino)ethyl]-1,3-dioxo-2,3-dihydro-1H-benzo[de]isoquinolin-5-yl]-N′-1,3-benzodioxol-5-yl-thiourea

200 mg of amonafide were dissolved in 4 mL of acetonitrile under nitrogen atmosphere. 273 mg of 1,3-benzodioxol-5-ylmethyl isothiocyanate (2 equivalents) in 4 mL of acetonitrile were carefully added. Reaction was maintained at room temperature for 5 minutes, then at 50° C. for 29 hours and at 65° C. for 21 hours. Acetonitrile was then evaporated under reduced pressure and the residue was submitted to a flash chromatography (SiO₂, eluent : CH₂Cl₂/MeOH 95:5), thus resulting in 205.4 mg (yield : 61%) of the desired product: 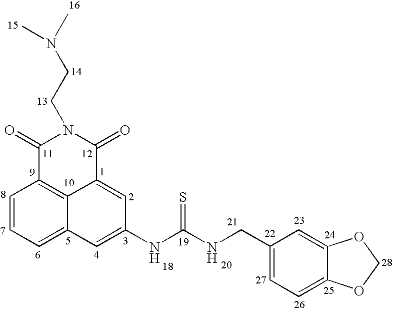
which was characterized by ¹H NMR (300 MHz, DMSO) as follows: 10.13 (H-18, bs); 8.3-8.7 (H-2, H-4, H-6, H-8 and H-20); 7.82 (H-7, t, J=7.6); 6.98 (H-27, bs); 6.89 (H-23 and H-26, bs); 6.00 (H-28, s); 4.16 (H-13, t, J=6.3); 2.50-2.55 (H-14, m) and 2.22 (H-15 and H-16, s).

### EXAMPLE 23

### In vitro Pharmacological Evaluation

In order to characterize the in vitro activity of the compounds of the invention, MTT tests were performed to indirectly and rapidly measure, i.e. within 5 days, the effect of such a compound on the overall cell growth. The test measures the number of metabolically active living cells that are able to transform the yellow product 3-(4,5-dimethylthiazol-2-yl)-2,5-diphenyl tetrazolium bromide (herein referred as MTT) into the blue product formazan dye by mitochondrial reduction. The amount of formazan obtained at the end of the experiment was measured by means of a spectrophotometer and is directly proportional to the number of living cells. Determination of the optical density enables a quantitative measurement of the effect of the investigated compounds as compared to the control condition (untreated cells) and/or to other reference compounds (in casu mitonafide and amonafide).

Six human cancer cell lines described in table 1 were used in the MTT tests. These cell lines cover four histological cancer types, being glioma, colon, lung and breast cancers. Cells were allowed to grow in 96-well micro-wells with a flat bottom with an amount of 100 μl of cell suspension per well with 1,000 to 4,000 cells/well depending on the cell type used. Each cell line was seeded in an MEM 5% serum culture medium.

TABLE 1

| | ATCC | | |
|---|---|---|---|
| Cell line | code | tissue | literature reference |
| Hs683 | HTB-138 | Glioma | J. Natl. Cancer Inst. 56: 843-849, 1976; |
| | | | ibid. 58: 1455-1463, 1977 |
| U-373MG | HTB-17 | Glioma | Acta Pathol. Microbial. Scand. |
| | | | 74: 465-486, 1968 |
| HCT-15 | CCL-225 | Colon | Cancer Res. 39: 1020-1025, 1979 |
| LoVo | CCL-229 | Colon | Exp. Cell Res. 101: 414-416, 1976; J. |
| | | | Natl. Cancer Inst. 61: 75-83, 1978; |
| | | | Cancer Res. 39: 2630-2636, 1979 |
| A549 | CCL-185 | Lung | J. Natl. Cancer Inst. 51: 1417-1423, |
| | | | 1973; Int. J. Cancer 17: 62-70, 1976 |
| MCF-7 | HTB-22 | Breast | J. Natl. Cancer Inst. 51: 1409-1416, |
| | | | 1973 |

The detailed experimental procedure was as follows: after a 24-hour period of incubation at 37° C., the culture medium was replaced by 100 μl of fresh medium in which the compound to be tested was dissolved at the following molar concentrations: 10⁻⁹ M, 5.10⁻⁹ M, 10⁻⁸ M, 5.10⁻⁸ M, 10⁻⁷ M, 5.10⁻⁷ M, 10⁻⁶ M, 5.10⁻⁶ M and 10⁻⁵ M. Each experiment was repeated 6 times.

After 72 hours of incubation at 37° C. with (experimental conditions) or without (control condition) the compound to be tested, the medium was replaced by 100 μl MTT dissolved in RPMI at a concentration of 1 mg/ml. The micro-wells were subsequently incubated during 3 hours at 37° C. and centrifuged at 400 g during 10 minutes. MTT was removed and formazan crystals formed were dissolved in 100 μl DMSO. The micro-wells were shaken for 5 minutes and read on a spectrophotometer at wavelengths of 570 nm (maximum formazan absorbance) and 630 nm (background noise).

For each experimental condition, the mean optical density was calculated, as well as the percentage of remaining living cells in comparison with the control.

Table 2 below shows the IC₅₀ values for compounds of examples 1 to 22 as well as for the closest prior art compounds, mitonafide and amonafide. This represents the range of molar concentrations of the compound tested that resulted in a 50% inhibition of overall tumor cells growth.

TABLE 2

| Compound | | Compound | |
|---|---|---|---|
| of example | IC₅₀ (M) | of example | IC₅₀ (M) |
| mitonafide | 5 · 10⁻⁶-10⁻⁶ | amonafide | 5 · 10⁻⁶-10⁻⁶ |
| 1 | 5 · 10⁻⁶-10⁻⁶ | 2 | 10⁻⁶-5 · 10⁻⁷ |
| 3 | 5 · 10⁻⁶-10⁻⁶ | 4 | 5 · 10⁻⁶-10⁻⁶ |
| 5 | 5 · 10⁻⁶-10⁻⁶ | 6 | 5 · 10⁻⁶-10⁻⁶ |
| 7 | 5 · 10⁻⁶-10⁻⁶ | 8 | 5 · 10⁻⁶-10⁻⁶ |
| 9 | 10⁻⁵-5 · 10⁻⁶ | 10 | 5 · 10⁻⁶-10⁻⁶ |
| 11 | >10⁻⁵ | 12 | >10⁻⁵ |
| 13 | 10⁻⁵-5 · 10⁻⁶ | 14 | >10⁻⁵ |
| 15 | 5 · 10⁻⁶-10⁻⁶ | 17 | >10⁻⁵ |
| 18 | >10⁻⁵ | 21 | 5 · 10⁻⁶-10⁻⁶ |
| 16 | >10⁻⁵ | 19 | 10⁻⁵ |
| 20 | 10⁻⁵-5 · 10⁻⁶ | 22 | >10⁻⁵ |

As a conclusion, most of the tested compounds exhibit an in vitro cytotoxic activity equivalent to that of mitonafide and amonafide.

### EXAMPLE 24

### In vivo Pharmacological Evaluation (Maximum Tolerated Dose)

The maximum tolerated dose (herein after MTD) is defined as the maximum amount of a given drug which can be administered acutely (i.e. in one intraperitoneal, intravenous, subcutaneous or oral single dose) to healthy animals, i.e. animals not grafted with tumors. The survival times and weights of the animals are recorded up to 14 days post injection. Five different doses of each drug are used for the determination of the MTD index. When said index is higher than 160 mg/kg (intraperitoneous administration) the drug is usually considered to be non-toxic, and the highest dose administered to tumor-bearing mice is MTD/2=80 mg/kg. Each experimental group comprised 3 mice for the determination of the MTD index (expressed in mg/kg). Using this methodology, the following data were obtained and reported in table 3. As a conclusion, the tested compounds are shown to be significantly less toxic than amonafide or mitonafide.

TABLE 3

| compound | | compound | |
|---|---|---|---|
| of example | MTD mg/kg | of example | MTD mg/kg |
| mitonafide | 40 | amonafide | 40 |
| 2 | >160 | 3 | 80 |
| 4 | 80 | 5 | >160 |
| 6 | >160 | 7 | >160 |
| 8 | >160 | 9 | >160 |
| 10 | >160 | 11 | >160 |
| 17 | 80 | 19 | 80 |

### EXAMPLE 25

### In vivo Pharmacological Evaluation—Mouse Leukemia Model

L1210 is a syngeneic model (Mouse Leukemia) which may be used to define an optimal treatment regimen (doses and schedule) for subsequent testing of drugs in more expensive and time-consuming orthotopic human xenograft models. The model was performed with 5 mice per group, said mice being grafted at day 0 and the tested compounds being injected intraperitoneally (at the dose indicated in table 4) at days 1, 2, 3, 4, 7, 8, 9 and 10 respectively. The data shown in table 4 are T/C values which were calculated by dividing the median day of death in a treated group T by the median day of death in the control group C. T/C values of 130% or more (i.e. a prolongation of mice survival of 30% or more) indicate a significant prolongation of survival.

TABLE 4

| | dose | | | dose | |
|---|---|---|---|---|---|
| compound | (mg/kg) | T/C (%) | compound | (mg/kg) | T/C (%) |
| mitonafide | 20 | 193 | amonafide | 20 | 120 |
| | 10 | 120 | | 10 | 120 |
| | 5 | 140 | | 5 | 140 |
| | 2.5 | 107 | | 2.5 | 120 |
| example 2 | 80 | 194 | example 7 | 80 | 213 |
| | 40 | 177 | | 40 | 147 |
| | 20 | 156 | | 20 | 147 |
| | 10 | 128 | | 10 | 140 |
| example 9 | 80 | 187 | example 10 | 80 | 194 |
| | 40 | 160 | | 40 | 139 |
| | 20 | 140 | | 20 | 156 |
| | 10 | 113 | | 10 | 117 |

As a conclusion, the tested compounds show a significant effect on prolongation of survival in the L-1210 model but at more and higher doses than mitonafide and amonafide.

### EXAMPLE 26

### Preparation of N-[2-[2-(dimethylamino)ethyl]-1,3-dioxo-2,3-dihydro-1H-benzo[de]isoquinolin-5-yl]-N′-(4-chlorophenyl)thiourea

300 mg of amonafide were dissolved in 6 mL of acetonitrile under nitrogen atmosphere. 360 mg of 4-chlorophenyl isothiocyanate (2 equivalents) in 6 mL of acetonitrile were carefully added. Reaction was maintained at room temperature for 5 minutes, then at 50° C. for 65 hours. Acetonitrile was then evaporated under reduced pressure and the residue was submitted to a flash chromatography (SiO₂, eluent: CH₂Cl₂/MeOH 90:10), thus resulting in 287.7 mg (yield: 60%) of the desired product: 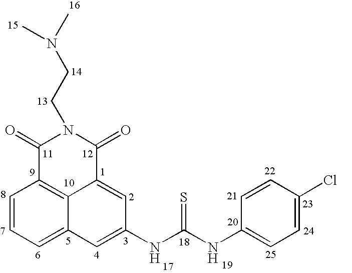
which was characterized by:
RMN ¹H (300 MHz, DMSO) as follows: 10.34 (H-17 and H-19, bs); 8.67 (H-2, d, J=2.1); 8.47 (H-4, d, J=2.1); 8.39 (H-8, d, J=6.6); 8.36 (H-6, d, J=7.2); 7.81 (H-7, t, J=7.8); 7.59 (H-21 and H-25, d, J=8.7); 7.42 (H-22 and H-24, d, J=8.4); 4.16 (H-13, t, J=6.7); 2.55 (H-14, t, J=7.2) and 2.24 (H-15 and H-16, s); and
RMN ¹³C (75.4 MHz, DMSO) as follows: 180.0 (C-18); 163.2 and 163.0 (C-11 and C-12); 138.5 and 138.1 (C-3 and C-20); 133.6; 131.6; 129.5; 128.5; 128.4 (C-22 and C-24); 128.3; 127.4; 126.0; 125.3 (C-21 and C-25); 124.6; 121.8; 121.7; 56.3 (C-14); 45.2 (C-15 and C-16) and 37.5 (C-13).

### EXAMPLE 27

### Preparation of N-[2-[2-(dimethylamino)ethyl]-1,3-dioxo-2,3-dihydro-1H-benzo[de]isoquinolin-5-yl]-N′-(4-cyanophenyl)thiourea

300 mg of amonafide were dissolved in 6 mL of acetonitrile under nitrogen atmosphere. 340 mg of 4-cyanophenyl isothiocyanate (2 equivalents) and 28 mg of aluminium chloride AlCl₃ (0.2 equivalents) in 6 mL of acetonitrile were carefully added. Reaction was maintained at room temperature for 5 minutes, then at 65° C. for 65 hours. Acetonitrile was then evaporated under reduced pressure and the residue was submitted to a flash chromatography (SiO₂, eluent : CH₂Cl₂/MeOH 95:5), thus resulting in 201.9 mg (yield =43%) of the desired product: 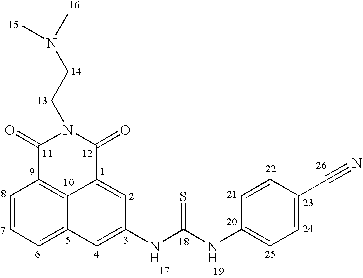
h was characterized by:
RMN ¹H (300 MHz, DMSO) as follows:11.23 (H-17 and H-19, bs); 8.73 (H-2, d, J=2.1); 8.57 (H-4, d, J=1.8); 8.40 (H-8, bs); 8.38 (H-6, bs); 7.96 (H-22 and H-24, d, J=9.0); 7.82 (H-7, t, J=7.8); 7.80 (H-21 and H-25, d, J=8.7); 4.18 (H-13, t, J=6.5); 2.62 (H-14, t, J=6.6) and 2.29 (H-15 and H-16, s), and
RMN ¹³C (75.4 MHz, DMSO) as follows : 178.9 (C-18); 163.3 and 163.0 (C-11 and C-12); 133.6; 132.7; 131.7; 129.4; 127.9; 127.4; 125.5; 124.6; 121.8; 121.7; 119.0; 105.1; 56.2 (C-14); 45.0 (C-15 and C-16) and 37.2 (C-13).

### EXAMPLE 28

### Preparation of 2-[2-dimethylamino)ethyl]-5-{[(1Z)-(4-cyano-phenyl)-5-ylmethylene]amino}-1H-benzo[de]isoquinolin-1,3(2H)-dione

1.0 g of amonafide, 33 mL of toluene and 556 mg 4-cyano-benzaldehyde (1.2 equivalent) were refluxed for 16 hours. After cooling, toluene was evaporated under reduced pressure and the residue was submitted to a flash chromatography (SiO₂, eluent : CH₂Cl₂/MeOH 95:5), thus resulting in 1.13 g of (yield =81%) of the desired product: 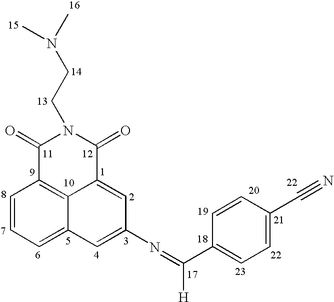
which was characterised by:
RMN ¹H (300 MHz, CDCl3) as follows: 8.71 (H-17, s); 8.54 (H-8, dd, J=6.3 and 1.0); 8.50 (H-2, d, J=2.4); 8.21 (H-6, dd, J=7.5 and 1.0); 8.09 (H-20 and H-22, d, J=8.4); 8.01 (H-4, d, J=2.1); 7.81 (H-19 and H-23, d, J=8.1); 7.76 (H-7, t, J=7.8); 4.34 (H-13, t, J=6.9); 2.67 (H-14, t, J=7.0) and 2.36 (H-15 and H-16, s);
RMN ¹³C (75.4 MHz, CDCl3) as follows: 163.9 and 163.8 (C-11 and C-12); 159.9 (C-17); 139.3 (C-3); 133.8; 132.6; 130.7; 129.3; 127.6; 127.3; 126.8; 125.4; 124.4; 123.9; 122.7; 118.2; 115.0; 56.3 (C-14); 45.3 (C-15 and C-16) and 37.5 (C-13); and
MS (EI) as follows: 58 (100); 71 (36) and 396 (1).

### EXAMPLE 29

### Preparation of 2,2,2-trifluoro-N-{2-[2-(dimethylamino)ethyl]-1,3-dioxo-2,3-dihydro-1H-benzo[de]isoquinolin-5-yl}acetamide

200 mg of amonafide were dissolved in 4 mL of acetonitrile under nitrogen atmosphere. 296 mg of trifluorocetic anhydride (2 equivalents) in 4 mL of acetonitrile were carefully added. Reaction was maintained at room temperature for 2 hours. Acetonitrile was then evaporated under reduced pressure and the residue was submitted to a flash chromatography (SiO₂, eluent : CH₂Cl₂/MeOH 90:10), thus resulting in 265.2 mg (yield : 99%) of the desired product: 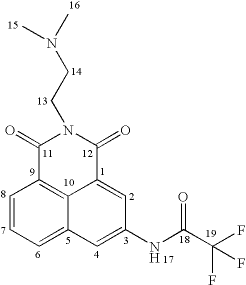
which was characterised by:
RMN ¹H (300 MHz, DMSO) as follows: 11.8 (H-17, bs); 8.78 (H-2, d, J=2.1); 8.75 (H-4, d, J=2.4); 8.46 (H-8, d, J=7.5); 8.44 (H-6, d, J=6.3); 7.87 (H-7, t, J=7.6); 4.33 (H-13, t, J=5.7); 3.23 (H-14, t, J=5.7) and 2.74 (H-15 and H-16, s); and
RMN ¹³C (75.4 MHz, DMSO) as follows: 163.5 and 163.2 (C-11 and C-12); 155.3 (C-18); 135.5 (C-3); 134.1; 131.6; 130.1; 127.9; 125.0; 124.5; 123.8; 122.9; 121.9; 117.5 (C-19); 55.1 (C-14); 43.3 (C-15 and C-16) and 35.7 (C-13).

### EXAMPLE 30

### Preparation of N-[({2-[2-(dimethylamino)ethyl]-1,3-dioxo-2,3-dihydro-1H-benzo[de]isoquinolin-5-yl]-N′-[4-methoxyphenyl]urea

200 mg of amonafide were dissolved in 4 mL of acetonitrile under nitrogen atmosphere. 210 mg of 4-methoxyphenylisocyanate (2 equivalents) in 4 mL of acetonitrile were carefully added. Reaction was maintained at room temperature for 16 hours. Acetonitrile was then evaporated under reduced pressure and the residue was submitted to a flash chromatography (SiO₂, eluent : CH₂Cl₂/MeOH 95:5), resulting in 259.1 mg (yield: 85%) of the desired product: 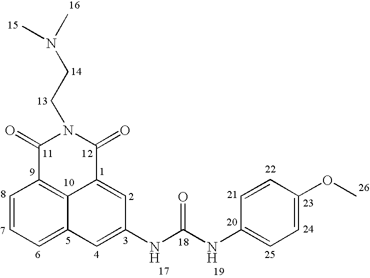
which was characterised by:
RMN ¹H (300 MHz, DMSO) as follows: 9.27 (H-19, s); 8.68 (H-17, s); 8.51 (H-2, d, J=1.2); 8.48 (H-4, d, J=2.1); 8.29 (H-8, d, J=0.9); 8.26 (H-6, bs); 7.75 (H-7, t, J=7.4); 7.43 (H-21 and H-25, d, J=9.0); 6.90 (H-22 and H-24, d, J=8.7); 4.14 (H-13, t, J=6.8), 2.45-2.55 (H-14, m) and 2.22 (H-15 and H-16, s); and
RMN ¹³C (75.4 MHz, DMSO) as follows: 163.3 and 163.1 (C-11 and C-12); 154.7 (C-23); 152.6 (C-18); 138.8 (C-3); 133.2; 132.2; 132.2; 128.2; 127.3; 123.3; 123.1; 122.3; 121.6; 120.3 (C-21 and C-25); 119.0; 113.9 (C-22 and C-24); 56.3 (C-14); 55.1 (C-26); 45.3 (C-15 and C-16) and 37.5 (C-13).

### EXAMPLE 31

### Preparation of N-[({2-[2-(dimethylamino)ethyl]-1,3-dioxo-2,3-dihydro-1H-benzo[de]isoquinolin-5-yl]-[4-methoxyphenyl]carbamate

200 mg of amonafide were dissolved in 4 mL of acetonitrile under nitrogen atmosphere. 269 mg of 4-methoxyphenyl chloroformate (2 equivalents) in 4 mL of acetonitrile were carefully added. Reaction was maintained at room temperature for 2 hours. Acetonitrile was then evaporated under reduced pressure and the residue was submitted to a flash chromatography (SiO₂, eluent: CH₂Cl₂/MeOH 90:10), resulting in 301.2 mg (yield : 98%) of the desired product: 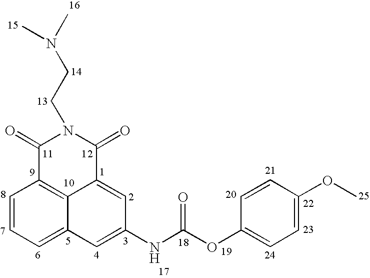
which was characterised by:
RMN ¹H (300 MHz, DMSO) as follows: 10.88 (H-17, bs); 8.62 (H-2, bs); 8.54 (H-4, bs); 8.35 (H-8, bs); 8.32 (H-6, bs); 7.79 (H-7, t, J=7.8); 4.26 (H-13, t, J=6.3); 2.94 (H-14, m) and 2.52 (H-15 and H-16, s); and
RMN ¹³C (75.4 MHz, DMSO) as follows: 163.5 and 163.2 (C-11 and C-12); 156.7 (C-18); 152.2 (C-22); 143.7 (C-19); 137.7 (C-3); 133.5; 131.9; 128.9; 127.6; 123.8; 123.2; 122.8; 122.7 (C-20 and C-24); 121.8; 119.8; 114.3 (C-21 and C-23); 55.4 (C-14 and C-25); 43.9 (C-15 and C-16) and 36.4 (C-13).

### EXAMPLE 32

### Preparation of 2-[2-(dimethylamino)ethyl]-1,3-dioxo-2,3-dihydro-1H-benzo[de]isoquinolin-5-ylbenzamide

200 mg of amonafide were dissolved in 4 mL of acetonitrile under nitrogen atmosphere. 200 mg of benzoyl chloride (2 equivalents) in 4 mL of acetonitrile were carefully added. Reaction was maintained at room temperature for 2 hours. Acetonitrile was then evaporated under reduced pressure and the residue was submitted to a flash chromatography (SiO₂, eluent: CH₂Cl₂/MeOH 90:10), resulting in 227.1 mg (yield: 83%) of the desired product: 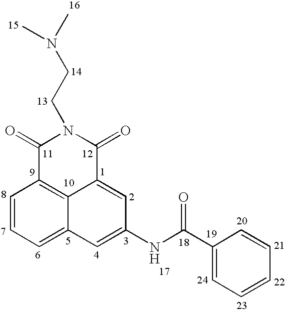
which was characterised by:
RMN ¹H (300 MHz, DMSO) as follows: 11.90 (H-17, bs); 8.99 (H-2, bs); 8.90 (H-4, bs); 8.40 (H-8, d, J=7.2); 8.38 (H-6, d, J=6.0); 8.09 (H-20 and H-24, d, J=7.2); 7.83 (H-7, t, J=7.5); 7.60 (H-21 and H-23, t, J=7.5); 7.63 (H-22, t, J=7.5); 4.16 (H-19, t, J=5.7); 2.61 (H-14, m) and 2.28 (H-15 and H-16, s); and
RMN ¹³C (75.4 MHz, DMSO) as follows: 165.9 (C-18); 163.3 and 163.2 (C-11 and C-12); 137.9 (C-3); 134.0; 133.7; 131.9; 129.1; 128.4; 127.8; 127.5; 125.0; 123.8; 122.4; 122.1; 121.7; 56.2 (C-14); 45.0 (C-15 and C-16) and 37.3 (C-13).

### EXAMPLE 33

### Preparation of 2,2,2-trichloro-N-[({2-[2-(dimethylamino)ethyl]-1,3-dioxo-2,3-dihydro-1H-benzo[de]isoquinolin-5-yl}amino)carbonyl]acetamide hydrochloride

6.3 g of the compound of example 2 were dissolved in 200 mL of ether, then 46 mmol of HCl (3.5 equivalents HCl, 3.7 mL of HCl 12.5 M) in 20 mL methanol were carefully added. The solid obtained was filtered and washed with ether, resulting in 4.864 g (yield: 72%) of the desired product: 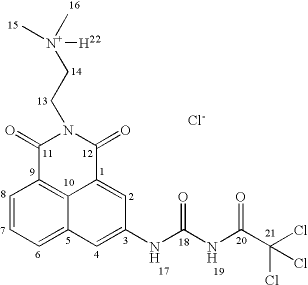
which was characterised by:
RMN ¹H (300 MHz, DMSO) as follows: 11.74 and 11.51 (H-17 and H-19, bs); 9.84 (H-22, bs); 863 (H-2, d, J=2.4); 8.60 (H-4, d, J=2.4); 8.41 (H-8, d, J=9.0); 8.39 (H-6, d, J=7.2); 7.84 (H-7, t, J=8.0); 4.40 (H-13, t, J=5.7), 3.47 (H-14, m) and 2.89 and 2.90 (H-15 and H-16, s); and
RMN ¹³C (75.4 MHz, DMSO): 163.8 and 163.5 (C-11 and C-12); 159.3 (C-18); 149.0 (C-20); 136.8; 133.8; 131.9; 129.2; 127.6; 127.3; 124.3; 123.5; 122.9; 121.1; 92.1 (C-21); 54.6 (C-14); 42.5 (C-15 and C-16) and 35.0 (C-13).

### EXAMPLE 34

### Preparation of 2-[2-dimethylamino)ethyl]-5-{[(1Z)-(2,5-dihydroxyphenyl) methylene]amino}-1H-benzo[de]isoquinolin-1,3(2H)-dione hydrochloride

1.20 g of the compound of example 19 were dissolved in 120 mL of ether, then 2.98 mmol of HCl (1 eq HCl, 238 μL of HCl 12.5 M) in 30 mL methanol were carefully added. The solid obtained was filtered and washed with ether, resulting in 1.21 g (yield: 93%) of the desired product: 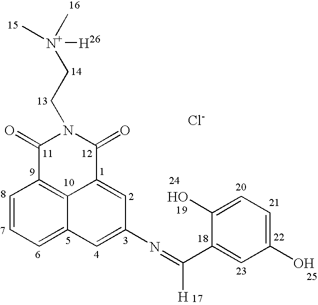
which was characterised by RMN ¹H (300 MHz, DMSO) as follows: 11.86 (H-17, bs); 9.20 and 9.10 (H-24 and H-25, bs); 8.47 (H-2, d, J=1.8); 8.45 (H-8, d, J=3.0); 8.42 (H-6, d, J=2.4); 8.38 (H-4, d, J=1.8); 7.87 (H-7, t, J=8.0); 7.17 (H-23, d, J=3.0); 6.92 (H-21, dd, J=3.0 and 8.7); 6.85 (H-20, d, J=8.7); 4.23 (H-13, t, J=6.5), 2.78 (H-14, m) and 2.40 (H-15 and H-16, s).

### EXAMPLE 35

### Preparation of 5-{[(1Z)-benzo([1,3]dioxol-6-nitro)-5-yl-methylene]amino}-N-(2-[2-dimethylamino)ethyl]-1H-benzo[de]isoquinolin-1,3(2H)-dione

300 mg of amonafide, 10 mL of toluene and 250 mg of 6-nitropiperonal (1.2 equivalent) were refluxed for 16 hours. After cooling, toluene was evaporated under reduced pressure and the residue was submitted to a flash chromatography (SiO₂, eluent : CH₂Cl₂/MeOH 95:5), resulting in 421.1 mg (yield : 86%) of the desired product: 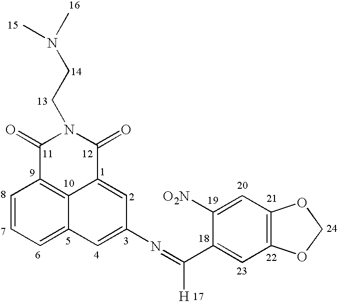
which was characterised by:
RMN ¹H (300 MHz, CDCl₃) as follows: 9.09 (H-17, s); 8.51 (H-2, d, J=2.1); 8.55 (H-8, dd, J=1.0 and 7.5); 8.22 (H-6, dd, J=1.0 and 8.4); 7.98 (H-4, d, J=2.1); 7.78 (H-7, t, J=4.0); 7.75 (H-20, d, J=7.2); 7.58 (H-23, s); 6.23 (H-24, s); 4.35 (H-13, t, J=6.9), 2.68 (H-14, t, J=7.2) and 2.37 (H-15 and H-16, s); and
RMN ¹³C (75.4 MHz, CDCl₃) as follows :_{—}164.1 and 163.8 (C-11 and C-12); 157.8 (C-17); 152.2; 150.2; 149.8; 144.9; 133.9; 132.6; 130.7; 127.7; 127.6; 126.9; 125.9; 124.0; 123.8; 122.7; 107.8; 105.3; 103.6 (C-24); 57.0 (C-14); 45.8 (C-15 and C-16) and 38.2 (C-13).

### EXAMPLE 36

### Preparation of 5-{[1,3-benzodioxol-5-ylmethyl]amino}-N-(2-[2-dimethylamino)ethyl]-1H-benzo[de]isoquinolin-1,3(2H)-dione

1.07 g of the compound of example 17, 15 mL of anhydrous methanol and 325 mg of NaBH₃CN (2 equivalents) were stirred for 2 hours. An aqueous sodium chloride solution was added and extracted with CH₂Cl₂. The organic phase was evaporated under reduced pressure and the residue was submitted to a flash chromatography (SiO₂, eluent: CH₂Cl₂/MeOH 90:10), followed by a second chromatograpy (RP-C18, eluent: MeOH/H₂O 90:10 to MeOH), thus resulting in 712.2 mg (yield=66%) of the desired product: 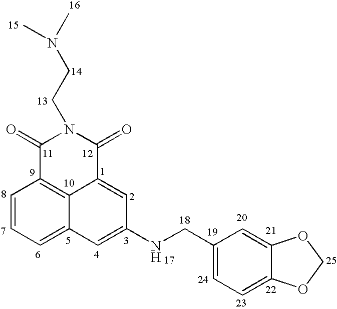
which was characterised by:
RMN ¹H (300 MHz, DMSO) as follows : 8.06 (H-8, d, J=8.4); 8.05 (H-2, d, J=2.4); 8.03 (H-6, d, J=9.0); 7.61 (H-7, t, J=7.7); 7.20 (H-4, d, J=2.1); 7.15 (H-17, t, J=6.0); 6.98 (H-20, bs); 6.92 (H-24, d, J=8.1); 6.87 (H-23, d, J=7.8); 5.97 (H-25, s); 4.12 (H-13, t, J=6.8), 2.50-2.55 (H-14, m) and 2.21 (H-15 and H-16, s); and
RMN ¹³C (75.4 MHz, DMSO) as follows 163.6 and 163.4 (C-11 and C-12); 147.3; 147.1; 146.0; 133.5; 132.8; 131.6; 127.0; 125.3; 122.3; 121.9; 121.6; 120.6; 120.3; 108.7; 108.0; 107.7; 100.7 (C-25); 56.4 (C-14); 45.9 (C-18); 45.2 (C-15 and C-16) and 37.3 (C-13).

### EXAMPLE 37

### Preparation of 5-{[2-(2-phenoxy-)-6-hydroxymethyltetrahydro-pyran-3,4,5-triol)-5-ylmethyl]amino}-N-(2-[2-dimethylamino)ethyl]-1H-benzo[de]isoquinolin-1,3(2H)-dione

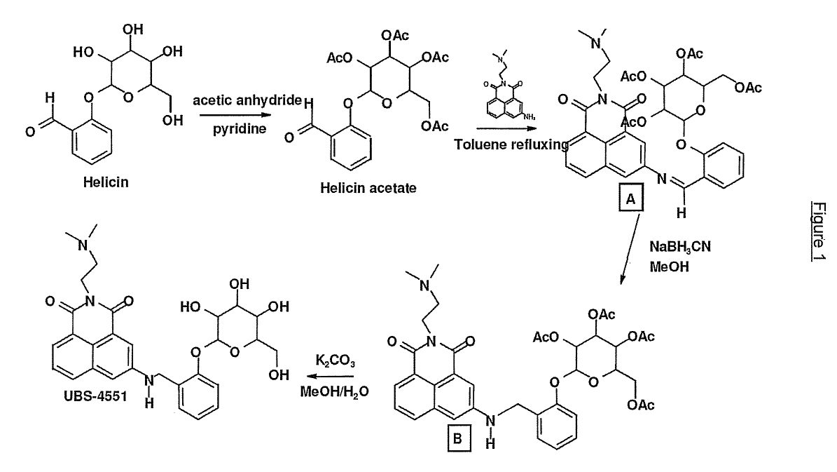

The synthetic route of this compound proceeds in a series of steps schematically shown in attached FIG. 1 and is described in more details as follows:
a) in a first step, 825 mg helicin and 8 mL of a pyridine/acetic anhydride 1:1 mixture were stirred for 16 hours. 8 mL of water at 0° C. was then added under stirring for 15 minutes. The solid obtained was filtered and washed with water, resulting in 1.15 g helicin acetate (yield : 88%);
b) in a second step, 600 mg amonafide, 15 mL toluene and 1.15 g helicin acetate (1.2 equivalent) from step (a) were refluxed for 16 hours. After cooling, toluene was evaporated under reduced pressure and the residue of the intermediate shown as product A in the figure (i.e. 5-{[(1Z)-benzo(3,5-bis(acetyloxy)-2-[(acetyloxy)methyl]-6-oxy-tetrahydro-2H-pyran-4-yl acetate)-5-ylmethylene] amino}-N-(2-[2-dimethylamino)ethyl]-1H-benzo [de]isoquinolin-1,3(2H)-dione) was directly submitted to the next reaction step;
c) 1.5 g of intermediate product A, 10 mL anhydrous methanol and 270 mg NaBH₃CN (2 equivalents) were stirred for 2 hours. An aqueous saturated sodium chloride solution was added and then extracted with CH₂Cl₂. The organic phase was evaporated under reduced pressure and the residue was submitted to a flash chromatography (SiO₂, eluent: CH₂Cl₂/MeOH 90:10), followed by a second chromatograpy (RP-C18, eluent: MeOH/H₂O 90:10 to MeOH), resulting in 738.5 mg of the intermediate shown as product B (i.e. 5-{[2-(2-phenoxy-)-6-hydroxymethyltetrahydropyran-3,4,5-triacetoxy)-5-ylmethyl]amino}-N-(2-[2-dimethylamino)ethyl]-1H-benzo[de]isoquinolin-1,3(2H)-dione) in the figure (yield: 49% for the combination of steps (b) and (c);
d) 727 mg of intermediate product B, 20 mL methanol, and 440 mg of K₂CO₃ (1 equivalent) in 1 mL of water were stirred for 15 minutes. Methanol was then evaporated under reduced pressure and the residue was submitted to a flash chromatography (SiO₂, eluent : CH₂Cl₂/MeOH 90:10), resulting in 536.1 mg (yield : 96%) of the desired product: 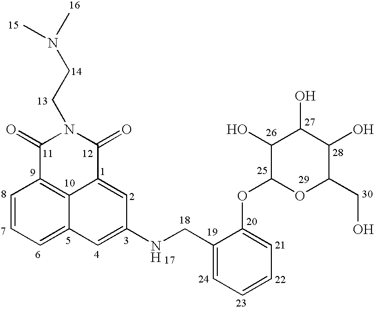
   which was characterised by:
   RMN ¹H (300 MHz, DMSO) as follows: 8.06 (H-2, s); 8.05 (H-4, s); 8.03 (H-8, d, J=3.3); 7.59 (H-7, t, J=7.8); 7.20 (H-6, d, J=2.7); 7.10 (H-17, t, J=6.3); 4.6-5.7 (4 OH, bs); 4.89 (H-25, d, J=7.5); 4.60 (1 H, dd, J=6.0 and 16.5); 4.43 (dd, J=6.9 and 16.2); 4.12 (H-13, t, J=6.7), 3.76 (1H, d, J=11.1); 3.52 (1 H, dd, J=5.7 and 11.7); 3.2-3.4 (4 H, m); 2.48 (H-14, t, J=6.6) and 2.19 (H-15 and H-16, s); and
   RMN ¹³C (75.4 MHz, DMSO) as follows: 163.6 and 163.4 (C-11 and C-12); 155.6 (C-20); 147.2; 133.6; 131.7; 128.0; 127.6; 126.9; 125.2; 122.3; 122.0; 121.7; 121.6; 120.5; 115.2; 108.6 (C-25); 101.5; 77.1; 76.5; 73.4; 69.7; 60.8 (C-30); 56.4 (C-14); 45.3 (C-15 and C-16); 40.9 (C-18) and 37.4 (C-13).

### EXAMPLE 38

### Preparation of 5-{[1,3-benzodioxol-5-ylmethy]amino}-N-(2-[2-dimethylamino)ethyl]-1H-benzo[de]isoquinolin-1,3(2H)-dione hydrochloride

250 mg of the compound of example 36 were dissolved in 10 mL of ether. 0.60 mmol of HCl (1 eq HCl, 48 pL of HCl 12.5 M) in 6 mL of MeOH were carefully added. Filter the solid and wash with ether, resulting in 266.4 mg (yield: 98%) of the desired compound: 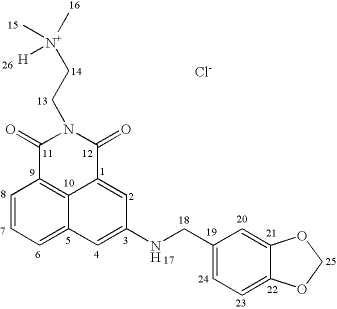
which was characterised by:
RMN ¹H (300 MHz, DMSO) as follows : 9.87 (H-17, bs); 8.09 (H-8, d, J=6.9); 8.07 (H-2, d, J=2.1); 8.05 (H-6, d, J=7.8); 7.63 (H-7, t, J=8.0); 7.24 (H-4, d, J=2.1); 6.98 (H-20, bs); 6.95 (H-24, d, J=8.0); 6.87 (H-23, d, J=8.1); 5.97 (H-25, s); 4.37 (H-13 and H-18, m); 3.41 (H-14, m) and 2.87 and 2.88 (H-15 and H-16, s); and
RMN ¹³C (75.4 MHz, DMSO) as follows: 164.1 and 163.9 (C-11 and C-12); 147.3; 147.0; 146.0; 133.4; 132.7; 131.6; 127.0; 125.3; 122.5; 122.0; 121.7; 120.8; 120.3; 108.5; 108.0; 107.7; 100.7 (C-25); 54.4 (C-14); 45.9 (C-18); 42.4 (C-15 and C-16) and 34.8 (C-13).

### EXAMPLE 39

### Preparation of 5-{[2-(2-phenoxy-)-6-hydroxymethyltetrahydro-pyran-3,4,5-triol)-5-ylmethyl]amino}-N-(2-[2-dimethylamino)ethyl]-1H-benzo [de]isoquinolin-1,3(2H)-dione hydrochloride

250 mg of the compound of example 37 were dissolved in 10 mL ether. 0.45 mmol of HCl (1 equivalent HCl, 36 pL of HCl 12.5 M) in 6 mL methanol were carefully added. The solid obtained was then filtered and washed with ether, resulting in 265.1 mg (yield: 99%) of the desired product: 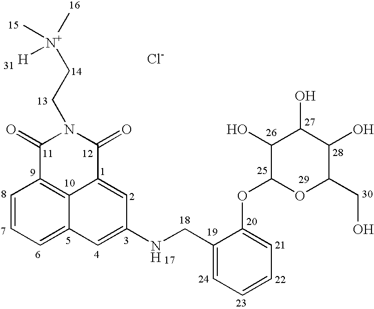
which was characterised by:
RMN ¹H (300 MHz, DMSO) as follows: 9.88 (H-17, bs); 8.09 (H-2, s); 8.09 (H-4, s); 8.06 (H-8, s); 7.62 (H-7, t, J=7.8); 7.34 (1H, d, J=7.5); 7.28 (1H, d, J=2.1); 7.19 (1H, m), 7.18 (H-6, s); 6.94(1 H, dt, J=2.4 and 8.1); 4.89 (H-25, d, J=6.9); 4.60 (1H, d, J=16.2); 4.43 (1 H, d, J=16.2); 4.36 (H-13, t, J=5.7), 3.74 (1 H, d, J=10.8); 3.5-4.5 (4 OH, bs); 3.52 (1 H, dd, J=6.0 and 12.0); 3.2-3.5 (6 H, m) and 2.87 and 2.88 (H-15 and H-16, s); and
RMN ¹³C (75.4 MHz, DMSO) as follows: 164.2 and 164.0 (C-11 and C-12); 155.6; 147.2; 133.6; 131.9; 128.0; 127.8; 127.6; 126.9; 125.2; 122.4; 122.0; 121.7; 121.6; 120.7; 115.2; 108.8 (C-25); 101.5; 77.1; 76.5; 73.4; 69.7; 60.7 (C-30); 54.5 (C-14); 42.5 and 42.5 (C-15 and C-16); 40.9 (C-18) and 34.9 (C-13).

### EXAMPLE 40

### Preparation of 5-{[(benzo([1,3]dioxol-6-nitro)-5-ylmethyl]amino}-N-(2-[2-dimethylamino)ethyl]-1H-benzo[de]isoquinolin-1,3(2H)-dione

The synthetic route of this compound proceeds in a series of two steps as follows:
a) in a first step, 1.0 g amonafide, 30 mL toluene and 830 mg of 6-nitropiperonal (1.2 equivalent) were refluxed for 16 hours. After cooling, toluene was evaporated under reduced pressure and the residue was directly submitted to the next reaction;
b) 1.62 g of the residue obtained in the first step, 10 mL anhydrous methanol and 445 mg NaBH₃CN (2 equivalents) were stirred for 2 hours. A saturated aqueous sodium chloride solution was added and then extracted with CH₂Cl₂. The organic phase was evaporated under reduced pressure and the residue was submitted to a flash chromatography (SiO₂, eluent : CH₂Cl₂/MeOH 90:10), thus resulting in 1.09 g (combined yield for reaction steps (a) and (b): 67%) of the desired product 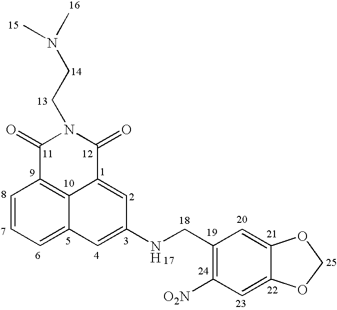
   which was characterised by:
   RMN ¹H (300 MHz, DMSO) as follows : 8.08 (H-2, d, J=2.4); 8.07 (H-8, d, J=6.9); 8.02 (H-6, d, J=7.8); 7.75 (H-23, s); 7.61 (H-7, t, J=7.7); 7.31 (H-17, t, J=5.9); 7.16 (H-4, d, J=2.4); 7.10 (H-20, s); 6.18 (H-25, s); 4.76 (H-18, d, J=6.0); 4.19 (H-13, t, J=6.6), 2.76 (H-14, m) and 2.40 (H-15 and H-16, s); and
   RMN ¹³C (75.4 MHz, DMSO) as follows: 163.8 and 163.5 (C-11 and C-12); 152.1; 146.6; 146.6; 141.7; 133.5; 132.5; 131.8; 127.0; 125.6; 122.6; 121.8; 121.6; 120.9; 108.6; 107.3; 105.7; 103.3 (C-25); 55.9 (C-14); 44.5 (C-15 ad C-16); 44.3 (C-18) and 36.7 (C-13).

### EXAMPLE 41

### Preparation of 5-{[(benzo([1,3]dioxol-6-nitro)-5-ylmethyl]amino}-N-(2-[2-dimethylamino)ethyl]-1H-benzo[de]isoquinolin-1,3(2H)-dione hydrochloride

250.3 mg of the compound of example 40 were dissolved in 10 mL ether. 0.54 mmol of HCl (1 equivalent HCl, 43 pL of HCl 12.5 M) in 5.5 mL methanol were carefully added. The solid obtained was filtered and washed with ether, resulting in 268.2 mg (yield 100%) of the desired product: 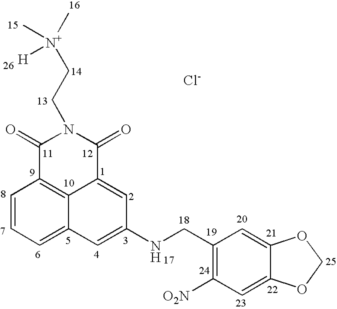
which was characterised by:
RMN ¹H (300 MHz, DMSO) as follows: 10.17 (H-17, bs); 8.11 (H-2, d, J=2.4); 8.09 (H-8, d, J=6.9); 8.03 (H-6, d, J=8.1); 7.75 (H-23, s); 7.62 (H-7, t, J=8.0); 7.18 (H-4, d, J=2.1); 7.10 (H-20, s); 6.18 (H-25, s); 4.76 (H-18, s); 4.38 (H-13, t, J=5.7), 3.42 (H-14, m) and 2.87 and 2.88 (H-15 and H-16, s); and
RMN ¹³C (75.4 MHz, DMSO) as follows: 164.1 and 163.9 (C-11 and C-12); 152.1; 146.6; 146.6; 141.7; 133.5; 132.5; 131.9; 127.0; 125.6; 122.7; 121.8; 121.8; 121.0; 108.7; 107.3; 105.7; 103.3 (C-25); 54.4 (C-14); 44.2 (C-18); 42.5 (C-15 ad C-16) and 34.9 (C-13).

### EXAMPLE 42

### Preparation of 5-{[2,3-dihydro-[1,4]-benzodioxin)-6-ylmethyl]amino}-N-(2-[2-dimethylamino)ethyl]-1H-benzo[de]isoquinolin-1,3(2H)-dione

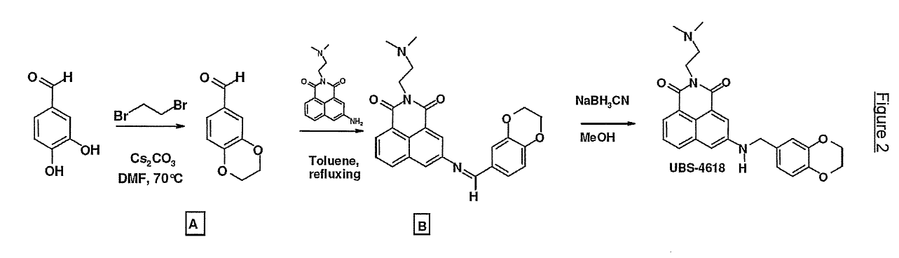

The synthetic route of this compound proceeds in a series of steps schematically shown in attached FIG. 2 and is described in more details as follows:
a) in a first step, 500 mg 3,4-dihydroxybenzaldehyde, 2.6 g cesium carbonate (2 equivalents), 1.4 g 1,2-dibromoethane (2 equivalents) and 5 mL anhydrous DMF were stirred at 70° C. for 16 hours. After cooling, the solvent was evaporated under reduced pressure and the residue was submitted to a flash chromatography (SiO₂, eluent : CH₂Cl₂), thus resulting in 575.3 mg (yield: 97%) of the intermediate shown as product A in FIG. 2;
b) 400 mg amonafide, 15 mL toluene and 280 mg of the intermediate product A (1.2 equivalent) were refluxed for 16 hours. After cooling, toluene was evaporated under reduced pressure and the residue of the intermediate shown as product B (i.e. 2-[2-dimethylamino)ethyl]-{[(1Z)-(2,3-dihydro-[1,4]-benzodioxin)-6-ylmethylene] amino}-1H-benzo[de]isoquinolin-1,3(2H)-dione) in FIG. 2 was directly submitted to the next reaction;
c) 610 mg of the intermediate product B, 10 mL anhydrous methanol and 177 mg NaBH₃CN (2 equivalents) were stirred for 2 hours. A saturated aqueous sodium chloride solution was added and then extracted with CH₂Cl₂. The organic phase was evaporated under reduced pressure and the residue was submitted to a flash chromatography (SiO₂, eluent: CH₂Cl₂/MeOH 90:10), followed by a second chromatograpy (RP-C18, eluent: MeOH/H₂O 80:20 to MeOH), thus resulting in 325 mg (combined yield for reactions (b) and (c) : 53%) of the desired product: 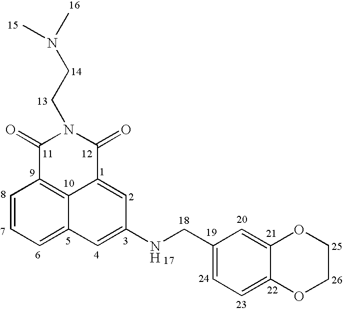
   which was characterised by:
   RMN ¹H (300 MHz, DMSO) as follows : 8.04 (H-8, d, J=6.3); 8.03 (H-2, d, J=2.7); 8.00 (H-6, d, J=7.5); 7.59 (H-7, t, J=7.8); 7.17 (H-4, d, J=2.7); 7.14 (H-17, t, J=6.0); 6.92 (H-20, s); 6.89 (H24, dd, J=2.1 and 10.2); 6.82 (H-23, d, J=8.4); 4.32 (H-18, d, J=5.7); 4.20 (s, H-25 and H-26); 4.12 (H-13, t, J=6.8), 2.50-2.55 (H-14, m) and 2.21 (H-15 and H-16, s); and
   RMN ¹³C (75.4 MHz, DMSO) as follows: 163.6 and 163.4 (C-11 and C-12); 147.1; 143.2; 142.2; 133.5; 131.9; 131.6; 127.0; 125.3; 122.3; 121.9; 121.6; 120.5; 120.0; 116.9; 115.8; 108.5, 64.0 and 63.9 (C-25 and C-26); 56.4 (C-14); 45.6 (C-18); 45.3 (C-15 and C-16) and 37.4 (C-13).

### EXAMPLE 43

### In vitro Pharmacological Evaluation

The compounds of examples 26 to 42 were tested according to the methodology described in example 23. Table 5 below shows the IC₅₀ values for these compounds, representing the range of molar concentrations of the compound tested that resulted in a 50% inhibition of overall tumor cells growth.

TABLE 5

| compound | | compound | |
|---|---|---|---|
| of example | IC₅₀ (M) | of example | IC₅₀ (M) |
| 26 | >10⁻⁵ | 27 | >10⁻⁵ |
| 28 | 10⁻⁵-5 · 10⁻⁶ | 29 | 10⁻⁵-5 · 10⁻⁶ |
| 30 | 5 · 10⁻⁶-10⁻⁶ | 31 | = 10⁻⁵ |
| | | 32 | >10⁻⁵ |
| 33 | 10⁻⁵-5 · 10⁻⁶ | 34 | 5 · 10⁻⁶-10⁻⁶ |
| 35 | >10⁻⁵ | 36 | 10⁻⁵-5 · 10⁻⁶ |
| 37 | 10⁻⁵-5 · 10⁻⁶ | 38 | 10⁻⁵-5 · 10⁻⁶ |
| 39 | >10⁻⁵ | 40 | >10⁻⁵ |
| 41 | 10⁻⁵-5 · 10⁻⁶ | 42 | 10⁻⁵-5 · 10⁻⁶ |

### EXAMPLE 44

### In vivo Pharmacological Evaluation (Maximum Tolerated Dose)

The maximum tolerated dose (herein after MTD) was determined according to the methodology of example 24. The data obtained for several compounds of the previous examples are reported in table 6.

TABLE 6

| compound | | compound | |
|---|---|---|---|
| of example | MTD mg/kg | of example | MTD mg/kg |
| 16 | >80 | 18 | >80 |
| 20 | >80 | 22 | >80 |
| 26 | >80 | 27 | >80 |
| 28 | >80 | 34 | 40 |
| 33 | 120 | 35 | 80 |
| 36 | >80 | 37 | >80 |
| 38 | >80 | 39 | >80 |
| 40 | >80 | 41 | >80 |
| 42 | >80 | | |

### EXAMPLE 45

### In vivo Pharmacological Evaluation—Mouse Leukemia Model

The experimental procedure of example 25 was repeated with compounds of examples 17, 19, 33 and 35, and the data obtained from this experiment are reported in table 7.

TABLE 7

| | dose | | | dose | |
|---|---|---|---|---|---|
| compound | (mg/kg) | T/C (%) | compound | (mg/kg) | T/C (%) |
| example 17 | 80 | 280 | example 19 | 80 | 275 |
| | 40 | 207 | | 40 | 231 |
| | 20 | 233 | | 20 | 144 |
| | 10 | 120 | | 10 | 150 |
| example 33 | 80 | 200 | example 35 | 80 | >300 |
| | 40 | 186 | | 40 | 280 |
| | 20 | 138 | | 20 | 193 |
| | 10 | 148 | | 10 | 213 |

As a conclusion, the tested compounds show a significant effect on prolongation of survival in the L-1210 model but (from a comparison with data presented in table 4) at higher doses than mitonafide and amonafide.

What is claimed is:

1. A substituted naphthalimide derivative represented by the general formula (I) 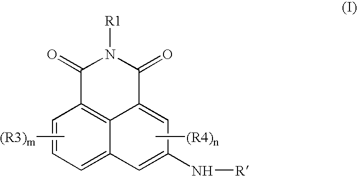
wherein
n=0, and/or
m=0, and/or
R₁ is alkylaminoalkyl, and/or
R′ is a radical selected from the group consisting of C₂₋₇ alkylcarbonyl, arylcarbonyl, arylalkylcarbonyl, alkylaminocarbonyl, arylaminocarbonyl, arylalkylamino-carbonyl, arylthioaminocarbonyl and arylalkylthioaminocarbonyl, wherein one or more carbon atoms of said radical are optionally substituted by one or more substituents independently selected from the group consisting of oxo, hydroxy, cyano, halogen, amino and nitro;
or by the general formula (II) 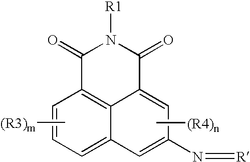
wherein:
m, n and R₁ are as defined with respect to formula (I), and
R′ is arylalkylidene, wherein one or more carbon atoms thereof are optionally substituted by one or more substituents independently selected from the group consisting of hydroxy, cyano, halogen, amino and nitro;
and/or a pharmaceutically acceptable salt thereof and/or a solvate thereof.

2. A substituted naphthalimide derivative according to claim 1, wherein R₁ is an alkylene radical having from 1 to 3 carbon atoms and being linked to dimethylamino, and/or a pharmaceutically acceptable salt thereof and/or a solvate thereof.

3. A substituted naphthalimide derivative selected from the group consisting of:
2-chloro-N-[({2-[2-(dimethylamino)ethyl]-1,3-dioxo-2,3-dihydro-1H-benzo[de]iso-quinolin-5-yl}amino)carbonyl]acetamide,
2,2,2-trichloro-N-[({2-[2-(dimethylamino)ethyl]-1,3-dioxo-2,3-dihydro-1H-benzo[de]isoquinolin-5-yl}amino)carbonyl]acetamide,
N-[({2-[2-(dimethylamino)ethyl]-1,3-dioxo-2,3-dihydro-1H-benzo[de]isoquinolin-5-yl}amino)carbonyl]benzamide,
ethyl({2-[2-(dimethylamino)ethyl]-1,3-dioxo-2,3-dihydro-1H-benzo[de]isoquinolin-5-yl}amino)carbonylcarbamate,
N-[2-[2-(dimethylamino)ethyl]-1,3-dioxo-2,3-dihydro-1H-benzo[de]isoquinolin-5-yl]-N′-(2-chloroethyl)urea,
N-[2-[2-(dimethylamino)ethyl]-1,3-dioxo-2,3-dihydro-1H-benzo[de]isoquinolin-5-yl]-N′-(4-chlorophenyl)urea,
N-[2-[2-(dimethylamino)ethyl]-1,3-dioxo-2,3-dihydro-1H-benzo[de]isoquinolin-5-yl]-N′-(4-cyanophenyl)urea,
ethyl 4-N-[({2-[2-(dimethylamino)ethyl]-1,3-dioxo-2,3-dihydro-1H-benzo[de]iso-quinolin-5-yl)amino]carbonyl}amino)benzoate,
N-[({2-[2-(dimethylamino)ethyl]-1,3-dioxo-2,3-dihydro-1H-benzo[de]isoquinolin-5-yl]-N′-1,3-benzodioxol-5-yl-urea,
N-[({2-[2-(dimethylamino)ethyl]-1,3-dioxo-2,3-dihydro-1H-benzo[de]isoquinolin-5-yl]-N′-[4-(trifluoromethoxy)phenyl]urea,
ethyl 4-N-[({2-[2-(dimethylamino)ethyl]1,3-dioxo-2,3-dihydro-1H-benzo[de]iso-quinolin-5-yl]amino}-4-oxobutanoate,
4-chloro-N-{2-[2-(dimethylamino)ethyl]-1,3-dioxo-2,3-dihydro-1H-benzo[de]iso-quinolin-5-yl}butanamide,
2-(4-chlorophenyl)-N-{2-[2-(dimethylamino)ethyl]-1,3-dioxo-2,3-dihydro-1H-benzo [de]isoquinolin-5-yl}acetamide,
ethyl 3-({2-[2-(dimethylamino)ethyl]-1,3-dioxo-2,3-dihydro-1H-benzo[de]isoquinolin-5-yl}amino)-3-oxopropanoate,
2-(4-methoxyphenyl)-N-{2-[2-(dimethyl-amino)ethyl]-1,3-dioxo-2,3-dihydro-1H-benzo[de]isoquinolin-5-yl}acetamide,
2,2,2-trichloro-N-{2-[2-(dimethylamino)ethyl]-1,3-dioxo-2,3-dihydro-1H-benzo[de]isoquinolin-5-yl}acetamide,
5-{[(1Z)-1,3-benzodioxol-5-ylmethylene]amino}-N-{-2-[2-(dimethylamino)ethyl]-1H-benzo[de]isoquinoline-1,3(2H)-dione,
5-{[(1Z)-(4-hydroxy-3-methoxyphenyl)methylene]amino}-2-[2-(dimethylamino) ethyl]-1H-benzo[de]isoquinoline-1,3(2H)-dione,
2-[2-(dimethylamino)ethyl]-5-{[(1Z)-(2,5-dihydroxyphenyl)methylene]amino)-1H-benzo[de]isoquinoline-1,3(2H)-dione,
2-[2-(dimethylamino)ethyl]-5-{[(1Z)-(3,4,5-trimethoxyphenyl)methylene]amino}-1H-benzo[de]isoquinoline-1,3(2H)-dione,
N-[({2-[2-(dimethylamino)ethyl]-1,3-dioxo-2,3-dihydro-1H-benzo[de]isoquinolin-5-yl]-N′-[4-(trifluoromethoxy)phenyl]thiourea, and
N-[({2-[2-(dimethylamino)ethyl]-1,3-dioxo-2,3-dihydro-1H-benzo[de]isoquinolin-5-yl]-N′-1,3-benzodioxol-5-ylmethyl-thiourea,
N-[2-[2-(dimethylamino)ethyl]-1,3-dioxo-2,3-dihydro-1H-benzo[de]isoquinolin-5-yl]-N′-(4-chlorophenyl)thiourea,
N-[2-[2-(dimethylamino)ethyl]-1,3-dioxo-2,3-dihydro-1H-benzo[de]isoquinolin-5-yl]-N′-(4-cyanophenyl)thiourea,
2-[2-dimethylamino)ethyl]-5-{[(1Z)-(4-cyano-phenyl)-5-ylmethylene]amino}-1H-benzo[de]isoquinolin-1,3(2H)-dione,
2,2,2-trifluoro-N-{2-[2-(dimethylamino)ethyl]-1,3-dioxo-2,3-dihydro-1H-benzo[de]isoquinolin-5-yl}acetamide,
N-[({2-[2-(dimethylamino)ethyl]-1,3-dioxo-2,3-dihydro-1H-benzo[de]isoquinolin-5-yl]-N′-[4-methoxyphenyl]urea,
N-[({2-[2-(dimethylamino)ethyl]-1,3-dioxo-2,3-dihydro-1H-benzo[de]isoquinolin-5-yl]-[4-methoxyphenyl]carbamate,
2-[2-(dimethylamino)ethyl]-1,3-dioxo-2,3-dihydro-1H-benzo[de]isoquinolin-5-ylbenzamide,
2,2,2-trichloro-N-[({2-[2-(dimethylamino)ethyl]-1,3-dioxo-2,3-dihydro-1H-benzo[de]isoquinolin-5-yl}amino) carbonyl]acetamide hydrochloride,
2-[2-dimethylamino)ethyl]-5-{[(1Z)-(2,5-dihydroxyphenyl)methylene]amino}-1H-benzo[de]isoquinolin-1,3(2H)-dione hydrochloride,
5-{[(1Z)-benzo([1,3]dioxol-6-nitro)-5-yl-methylene]amino}-N-(2-[2-dimethylamino)ethyl]-1H-benzo[de]isoquinolin -1,3(2H)-dione,
5-{[1,3-benzodioxol-5-ylmethyl]amino}-N-(2-[2-dimethylamino)ethyl]-1H-benzo[de]isoquinolin-1,3(2H)-dione,
5-{[2-(2-phenoxy-)-6-hydroxymethyltetrahydro-pyran-3,4,5-triol)-5-ylmethyl]amino}-N-(2-[2-dimethylamino)ethyl]-1H-benzo[de]isoquinolin-1,3(2H)-dione,
5-{[(1Z)-benzo(3,5-bis(acetyloxy)-2-[(acetyloxy)methyl]-6-oxy-tetrahydro-2H-pyran-4-yl acetate)-5-ylmethylene]amino}N-(2-[2-dimethylamino)ethyl]-1H-benzo [de]isoquinolin-1,3(2H)-dione,
5-{[2-(2-phenoxy-)-6-hydroxymethyltetrahydropyran-3,4,5-triacetoxy)-5-ylmethyl]amino}-N-(2-[2-dimethylamino)ethyl]-1H-benzo[de]isoquinolin-1,3(2H)-dione,
5-{[1,3-benzodioxol-5-ylmethyl]amino}-N-(2-[2-dimethylamino)ethyl]-1H-benzo[de]isoquinolin-1,3(2H)-dione hydrochloride,
5-{[2-(2-phenoxy-)-6-hydroxymethyltetrahydro-pyran-3,4,5-triol)-5-ylmethyl]amino}-N-(2-[2-dimethylamino)ethyl]-1H-benzo [de]isoquinolin-1,3(2H)-dione hydrochloride,
5-{[(benzo([1,3]dioxol-6-nitro)-5-ylmethyl]amino}-N-(2-[2-dimethylamino)ethyl]-1H-benzo[de]isoquinolin-1,3(2H)-dione,
5-{[(benzo([1,3]dioxol-6-nitro)-5-ylmethyl]amino}-N-(2-[2-dimethylamino)ethyl]-1H-benzo[de]isoquinolin-1,3(2H)-dione hydrochloride,
5-{[2,3-dihydro-[1,4]-benzodioxin)-6-ylmethyl]amino}-N-(2-[2-dimethylamino)ethyl]-1H-benzo[de]isoquinolin-1,3(2H)-dione, and
2-[2-dimethylamino)ethyl]-{[(1Z)-(2,3-dihydro-[1 ,4]-benzodioxin)-6-ylmethylene]amino}-1H-benzo[de]isoquinolin-1,3(2H)-dione,
and/or a pharmaceutically acceptable salt thereof and/or a solvate thereof.

4. A pharmaceutical composition comprising one or more pharmaceutically acceptable carriers and a therapeutically effective amount of a substituted naphthalimide derivative according to claim 1.

5. A pharmaceutical composition comprising one or more pharmaceutically acceptable carriers and a therapeutically effective amount of a substituted naphthalimide derivative according to claim 1, and further comprising an antineoplastic drug.

6. A pharmaceutical composition comprising one or more pharmaceutically acceptable excipients or carriers and a therapeutically effective amount of a substituted naphthalimide derivative according to claim 3.

7. A pharmaceutical composition comprising one or more pharmaceutically acceptable excipients or carriers and a therapeutically effective amount of a substituted naphthalimide derivative according to claim 3, and further comprising an antineoplastic drug.

8. A method of treatment of a cell proliferative disorder, comprising the administration to a patient in need thereof of a substituted naphthalimide derivative and/or a pharmaceutically acceptable salt thereof and/or a solvate thereof according to claim 1.

9. A method of treatment of a cell proliferative disorder comprising the administration, to a patient in need thereof, of a substituted naphthalimide derivative and/or a pharmaceutically acceptable salt thereof and/or a solvate thereof according to claim 1, wherein said naphthalimide derivative is admixed with one or more pharmaceutically acceptable excipients or carriers.

10. A method of treatment of a cell proliferative disorder, comprising the administration to a patient in need thereof of a substituted naphthalimide derivative and/or a pharmaceutically acceptable salt thereof and/or a solvate thereof according to claim 1, wherein said cell proliferative disorder is selected from the group consisting of leukemia, lung cancer, colorectal cancer, central nervous system (CNS) cancer, melanoma, ovarian cancer, kidney cancer, prostate cancer, breast cancer, glioma, bladder cancer, bone cancer, sarcoma, head and neck cancer, liver cancer, testicular cancer, pancreatic cancer, stomach cancer, oesophaegal cancer, bone marrow cancer, duodenum cancer, eye cancer (retinoblastoma) and lymphoma.

11. A method of treatment of a cell proliferative disorder, comprising the administration to a patient in need thereof of a substituted naphthalimide derivative and/or a pharmaceutically acceptable salt thereof and/or a solvate thereof according to claim 3.

12. A method of treatment of a cell proliferative disorder, comprising the administration to a patient in need thereof of a substituted naphthalimide derivative and/or a pharmaceutically acceptable salt thereof and/or a solvate thereof according to claim 3, wherein said naphthalimide derivative is admixed with one or more pharmaceutically acceptable excipients or carriers.

13. A method of treatment of a cell proliferative disorder, comprising the administration to a patient in need thereof of a substituted naphthalimide derivative, and/or a pharmaceutically acceptable salt thereof and/or a solvate thereof, according to claim 3, wherein said cell proliferative disorder is selected from the group consisting of leukemia, lung cancer, colorectal cancer, central nervous system (CNS) cancer, melanoma, ovarian cancer, kidney cancer, prostate cancer, breast cancer, glioma, bladder cancer, bone cancer, sarcoma, head and neck cancer, liver cancer, testicular cancer, pancreatic cancer, stomach cancer, oesophaegal cancer, bone marrow cancer, duodenum cancer, eye cancer (retinoblastoma) and lymphoma.

14. A method of making a substituted naphthalimide derivative according to claim 1, said substituted naphthalimide derivative being represented by the general formula (I), said method comprising reacting a N-(R₁-substituted)-3-amino-1,8-naphthalimide optionally having m substituents R₃ and/or n substituents R₄ with an R′-containing reagent being able to react with the 3-amino group of the naphthalimide derivative without substantially reacting with other substituents that may be present on the naphthalimide ring, wherein said R′-containing reagent is selected from the group consisting of acyl halides, thioacyl halides, monoisocyanates, isothiocyanates and polyamines.

15. A method of making a substituted naphthalimide derivative according to claim 1, said substituted naphthalimide derivative being represented by the general formula (II), said method comprising reacting a N-(R₁-substituted)-3-amino-1,8-naphthalimide optionally having m substituents R₃ and/or n substituents R₄ with an aldehyde having the formula R′CH(O).
